(19) United States
(12) Patent Application Publication
(10) Pub. No.: US 2025/0152919 A1
(43) Pub. Date: May 15, 2025

(54) RESPIRATORY THERAPY ACCESSORY

(71) Applicant: Fisher & Paykel Healthcare Limited, Auckland (NZ)

(72) Inventors: Andrew William WHITE, Auckland (NZ); Mehak Iqbal JANJUA, Auckland (NZ); Emeric Henri Michel BELLEUX, Auckland (NZ); Hugo Max Treherne JAMES, Auckland (NZ); Jason Allan KLENNER, Auckland (NZ); Mustafa Basil TAHA, Auckland (NZ)

(21) Appl. No.: 18/685,827

(22) PCT Filed: Aug. 25, 2022
(86) PCT No.: PCT/NZ2022/050113

§ 371 Date: Feb. 22, 2024

Related U.S. Application Data
(60) Provisional application No. 63/269,535, filed on Mar. 17, 2022.

(30) Foreign Application Priority Data
Aug. 25, 2021 (AU) 2021221574

(51) Int. Cl. A61M 25/02

(52) U.S. Cl. CPC A61M 25/02; A61M 2025/024; A61M 2025/028

(57) ABSTRACT

A respiratory therapy accessory comprises a body configured to be coupled to a support. The respiratory therapy accessory comprises at least one retaining member extending from the body. The retaining member is configured to retain a respiratory therapy component. In some examples, the respiratory therapy accessory comprises three retaining members. The three retaining members may be structurally dissimilar and extend from the body in different directions. The body may be configured to attach to the support by translation and rotation of the respiratory therapy accessory as a whole. The body may be configured to slide or rotate relative to the support. The respiratory therapy accessory may be used to facilitate transitioning a patient from one form of respiratory therapy to another, which involves changing at least the patient interface assembly used by the patient.

This application claims the benefit of Australian patent application No. 2021221574, filed on Aug. 25, 2021, and U.S. Provisional patent application Ser. No. 63/269,535, filed on Mar. 17, 2022. The disclosure of each of these prior applications is incorporated herein by reference in its entirety.

### BACKGROUND

### Field

The present disclosure relates to accessories for respiratory therapy and respiratory therapy systems including such an accessory. More particularly, though not exclusively, the disclosure relates to a respiratory therapy accessory configured to attach to a support and retain at least one respiratory therapy component.

### Description of Related Art

Respiratory apparatuses are used in various environments, such as hospital, medical facilities, residential care, palliative care and home environments. For a range of respiratory applications, it is beneficial to humidify breathing gases being supplied to a patient from a gases source. Humidifying the gases increases patient comfort, improves the patient's tolerance to the therapy, and the humidified gases are less prone to drying out the tissues (for example, the nasal mucosa) of the airway of the patient.

Respiratory therapy may be provided in several different forms, including at least invasive ventilation, non-invasive ventilation (NIV) and nasal high flow (NHF) therapy.

In the case of invasive ventilation therapy, humidified breathing gases are delivered to a patient via an invasive patient interface assembly which bypasses the patient's upper airways. The invasive patient interface assembly may be an intubated endotracheal tube, or a tracheostomy tube. The gases source may be a ventilator which delivers pressurized gases to a humidifier through a “dry line” conduit. The humidifier heats and humidifies the gases which are then conveyed to the patient interface assembly by an inspiratory conduit. Expired gases may be vented to atmosphere (referred to as a single-limb circuit) or returned to the ventilator via an expiratory conduit fluidly coupled with the inspiratory conduit and the invasive patient interface assembly via a wye-piece (referred to as a dual-limb circuit).

In the case of non-invasive ventilation (NIV) therapy, the breathing gases are delivered to the patient's upper airway via a non-invasive patient interface assembly. The non-invasive patient interface assembly may include a total face mask (sealing around the patient's nose, mouth and eyes), a full-face mask (sealing around the patient's nose and mouth) or an oro-nasal mask (sealing with or around each of the patient's nose/nares and mouth individually). A non-invasive patient interface assembly sealing with the patient's nasal passages may be suitable for some patients. This may be in the form of a nasal mask (sealing around the patient's nose) or a nasal pillows mask (sealing with each of the patient's nares). The gases source may be the same as that used for invasive ventilation therapy, if configured to operate in both invasive and non-invasive therapy modes. For the purposes of this disclosure, NIV treatment is intended to encompass bi-level ventilation and continuous positive airway pressure (CPAP) modes.

In the case of nasal high flow (NHF) therapy, humidified gases are delivered to the patient at relatively high flow rates through a non-sealing patient interface assembly such as a nasal cannula assembly which delivers the gases directly into the patient's nares through a pair of non-sealing prongs. The patient may be spontaneously breathing or may be apneic, such as under anesthesia. A gases source with a humidifier can be used to deliver the high flow gases. The gases source and humidifier may be the same as that used for invasive or non-invasive therapy, if configured to further operate in an NHF mode, or the gases source and humidifier may be integrated in a single flow therapy device. The flow therapy device may have an integrated flow generator such as a blower which generates the flow of gases from ambient air. The gases source and/or humidifier may control characteristics of the gases flow, such as one or more of flow rate, temperature, pressure, humidity, supplementary gases concentration, and the like. The flow of gases may be delivered to the nasal cannula assembly by an inspiratory conduit in a single-limb circuit. Expired gases may be exhaled through the patient's nares around the prongs of the nasal cannula assembly, or through the patient's mouth.

Changing a patient between different forms of respiratory therapy requires changing at least the patient interface assembly (e.g., the aforementioned invasive, non-invasive and non-sealing patient interface assemblies), and may further require changing one or more of the inspiratory conduit, expiratory conduit and gases source.

### SUMMARY

In a first aspect, a respiratory therapy accessory includes: a body configured to be coupled to a support, the body including a longitudinal axis; a first retaining member configured to hold a first respiratory therapy component; a second retaining member configured to hold a second respiratory therapy component; and a third retaining member configured to hold a third respiratory therapy component; wherein the first retaining member, the second retaining member, and the third retaining member each differ in shape and extend from the body in different directions.

Two or more of the first respiratory therapy component, the second respiratory therapy component and the third respiratory therapy component may be configured for use in different forms of respiratory therapy. The different respiratory therapies may include any two or more of invasive ventilation, non-invasive ventilation (NIV) and nasal high flow (NHF) therapy.

The first retaining member, the second retaining member and the third retaining member may extend from the body in respective directions which, when viewed along the longitudinal axis, together span an angle of no more than about 180°. In some examples, the angle may be about 140°. In other examples, the angle may be no more than about 120°, or no more than about 90°. The angle may be defined between the first retaining member and the second retaining member.

The third retaining member may extend from the body in a direction which bisects the angle defined between the first retaining member and the second retaining member.

Any one or more of the first retaining member, the second retaining member and the third retaining member may extend in a direction perpendicular to the longitudinal axis of the body.

Any one or more of the first retaining member, the second retaining member and the third retaining member may extend in a radial direction with respect to the longitudinal axis.

Each of the first retaining member, second retaining member and third retaining member may extend in a respective radial direction with respect to the longitudinal axis.

The first retaining member may be spaced apart from the second retaining member in a direction of the longitudinal axis.

At least one of the first retaining member and the second retaining member may include a part annular portion configured to hold the first respiratory therapy component or the second respiratory therapy component, respectively.

The third retaining member may include a hook.

The third retaining member in cross-section may have a width that is greater than a thickness of the third retaining member.

The first respiratory therapy component may include a non-sealing nasal cannula assembly.

The second respiratory therapy component may include a respiratory conduit.

The second retaining member may be configured to retain both the respiratory conduit and a further respiratory conduit, a maximum outer diameter of the respiratory conduit being different to a maximum outer diameter of the further respiratory conduit.

The second retaining member may be configured to retain only one of the respiratory conduit and the further respiratory conduit at a time. Alternatively, the second retaining member may be configured to retain both the respiratory conduit and the further respiratory conduit simultaneously.

The third respiratory therapy component may include an invasive patient interface assembly or a non-invasive patient interface assembly.

The third respiratory therapy component may include a patient interface assembly, and the patient interface assembly may include any one of a total face mask, a full-face mask, an oro-nasal mask, a nasal mask, a nasal pillows mask, an endotracheal tube, and a tracheostomy tube.

The first retaining member may include a pair of retaining arms defining a part annular portion configured to hold the first respiratory therapy component.

The second retaining member may include a pair of retaining arms configured to hold the second respiratory therapy component, the pair of retaining arms converging towards one another at a waist region intermediate respective distal and proximal ends of the pair of retaining arms, such that the pair of retaining arms define a first retaining space and a second retaining space, and at least one of the first retaining space and the second retaining space may be configured to hold the second respiratory therapy component.

The second retaining member may have a substantial figure-eight shape.

The third retaining member may be longer than the first retaining member and the second retaining member.

Any one or more of the first retaining member, the second retaining member and the third retaining member may be supported by a respective support member. The support member may include a strut or a gusset.

The first retaining member may include a part annular portion configured to hold the first respiratory therapy component, and a gusset extending from the body to a proximal end of the part annular portion of the first retaining member.

The second retaining member may include a pair of retaining arms defining a first retaining space and a second retaining space, and a gusset extending from the body to a proximal end of the pair of retaining arms of the second retaining member.

The third retaining member may include a hook at a distal end of the third retaining member, and a gusset extending from the body to a proximal end of the hook of the third retaining member.

A portion of the body may include a thickened region.

The thickened region may be thickest where the first retaining member extends from the body.

The thickened region may taper along at least a portion of the body in a longitudinal direction.

The thickened region may taper around at least a portion of the body in a circumferential direction.

The body may define an interior space and include: a first arm extending in a circumferential direction, the first arm defining a first opening to the interior space, and a second arm extending in an opposing circumferential direction, the second arm defining a second opening to the interior space, wherein the first arm and the second arm together define a slot extending between, and connecting, the first opening and the second opening, the first arm may be configured to engage a first portion of the support, and the second arm may be configured to engage a second portion of the support.

The first arm and the second arm may be resiliently deformable.

At least one of the first arm and the second arm may be configured to provide at least one of tactile and audible feedback upon coupling of the respiratory therapy accessory with the support, in use.

The first opening and the second opening may be located at opposing sides and ends of the body.

The first arm and the second arm may each be cantilevered from opposing sides of a spine of the body. The spine may be formed by a front portion of the body.

The first arm and the second arm may be part annular.

Two or more of the first retaining member, the second retaining member and the third retaining member may extend from the second arm.

The two or more of the first retaining member, the second retaining member and the third retaining member may be spaced an equal distance from at least one end of the body.

The first retaining member, the second retaining member and the third retaining member may each be spaced an equal distance from at least one end of the body.

The body may include a thickened region located between the first arm and the second arm.

In a second aspect, a method of coupling the respiratory therapy accessory of the first aspect with the support includes: aligning the slot with the support such that the longitudinal axis of the body is angled with respect to a longitudinal axis of the support, translating the body so that the support is partially received within the interior space, through the slot, and engaging the first arm and the second arm with the support by rotating the body such that the longitudinal axis of the body is parallel or coaxial with the longitudinal axis of the support, wherein the first arm and second arm deform as the support moves through the first opening and the second opening, and wherein the first arm grips the first portion of the support and the second arm grips the second portion of the support when the longitudinal axis of the body is parallel or axially aligned with the longitudinal axis of the support.

The method may further include rotating the body about a longitudinal axis of the support, and/or sliding the body along the longitudinal axis of the support.

The method may further include hanging a respiratory therapy component from at least one of the first retaining member, the second retaining member, and the third retaining member.

The method may further include: hanging a non-sealing nasal cannula assembly from the first retaining member, hanging a respiratory conduit from the second retaining member, and/or hanging an invasive patient interface assembly or a non-invasive patient interface assembly from the third retaining member.

The step of engaging the first arm and the second arm with the support may include rotating the body by moving a distal end of the third retaining member downwardly.

The step of engaging the first arm and the second arm with the support may include moving the third retaining member to a position which is perpendicular to the support.

In a third aspect, a respiratory therapy accessory includes a body configured to be coupled to a support, the body defining an interior space and including: a first arm extending in a circumferential direction, the first arm defining a first portion of the interior space and a first opening to the interior space adjacent a distal end of the first arm; a second arm extending in an opposing circumferential direction, the second arm defining a second portion of the interior space and a second opening to the interior space adjacent a distal end of the second arm, and the first arm and the second arm together defining a slot extending between, and connecting, the first opening and the second opening; and one or more retaining members extending outwardly from the body, the one or more retaining members each configured to retain a respiratory therapy component.

A width of the first opening may be less than a diameter of the first portion of the interior space and/or a width of the second opening may be less than a diameter of the second portion of the interior space.

At least one of the first opening and the second opening may be defined by opposing edges of the body, wherein the opposing edges are parallel with a longitudinal axis of the body, and the opposing edges are configured to determine, in part, a torque required to rotate the body onto the support, such that the support is retained within the interior space.

At least one of the first arm and the second arm may be configured to elastically deform as the support is rotated from a first orientation, in which the slot is configured to receive the support, to a second orientation, in which the support is received by the first portion and the second portion of the interior space.

At least one of the first arm and the second arm may be configured to elastically deform and partially recover when the body is coupled to the support.

A diameter of at least one of the first portion of the interior space and the second portion of the interior space may be equal to or less than a diameter of the support when the body is not coupled to the support.

A width of the slot may be equal to or greater than a diameter of the support.

At least one of the one or more retaining members may be configured to be grasped by a user to rotate the body around the support.

The first arm and the second arm may each extend through an angle of more than 180°.

The body may be configured to couple with the support so that it may selectively slide along and rotate about the support.

The respiratory therapy component may be one or more of a non-sealing nasal cannula assembly, a respiratory conduit, an invasive patient interface assembly, and a non-invasive patient interface assembly.

The respiratory therapy accessory may include two or more retaining members, each of the retaining members configured to retain a respiratory therapy component configured for use in a different form of respiratory therapy.

The first arm and the second arm may be part annular.

At least one of the one or more retaining members may extend from a side of the body opposing the first opening or the second opening.

At least one of the one or more retaining members may extend from the body in a direction that is perpendicular to the first opening and the second opening when viewed along a longitudinal axis of the body.

The one or more retaining members may include a plurality of divergent elongate arms.

In a fourth aspect, a method for attaching the respiratory therapy accessory of the third aspect to a support may include steps of: translating the body towards the support so that the support is partially received within the interior space through the slot, and rotating the body to engage the first arm and the second arm with the support, wherein the support is received within the first portion of the interior space through the first opening and within the second portion of the interior space through the second opening.

The method may include rotating the body about a longitudinal axis of the support, and/or sliding the body along the longitudinal axis of the support.

The method may include a step of hanging a respiratory therapy component from at least one of the one or more retaining members.

In a fifth aspect, a respiratory therapy accessory includes a body configured to be coupled to a support, the body including a longitudinal axis; a first retaining member extending from the body, the first retaining member defining a first interior space for receiving a connector of a patient interface assembly; a second retaining member extending from the body, the second retaining member defining a second interior space for receiving a respiratory conduit; a first ledge extending from the first retaining member towards the first interior space; and a second ledge extending from the second retaining member towards the second interior space; wherein a top surface of the first ledge is configured to abut a shoulder of the connector when the connector is received within the first interior space, such that the connector is supported by the first ledge and the patient interface assembly hangs from the first retaining member, in use, and the second ledge is configured to engage an outer surface of the respiratory conduit when the respiratory conduit is received within the second interior space, such that the respiratory conduit hangs from the second retaining member, in use.

The second ledge may be configured to engage an undulation in the outer surface of the respiratory conduit. The undulation may include a corrugation and the second ledge may be configured to be received between adjacent corrugations of the respiratory conduit.

The first retaining member may be spaced apart from the second retaining member in a direction of the longitudinal axis.

At least one of the first retaining member and the second retaining member may extend from the body in a direction perpendicular to the longitudinal axis.

At least one of the first retaining member and the second retaining member may extend from the body radially with respect to the longitudinal axis.

The first retaining member may include an arcuate wall extending upwardly from the first ledge, and the arcuate wall and the first ledge in combination may be configured to cradle the connector.

The arcuate wall may have an internal diameter that is equal to or greater than an external diameter of the shoulder.

The patient interface may include a non-sealing nasal cannula.

The first retaining member may extend from the body in a different direction to the second retaining member, such that the patient interface assembly is spaced apart from the respiratory conduit received within the second interior space, in use.

The second retaining member may further define a third interior space, the third interior space including a larger area than the second interior space, the third interior space configured to receive a second respiratory conduit including a larger maximum outer diameter than the respiratory conduit configured to be received within the second interior space.

The second retaining member may be configured to receive only one of the respiratory conduit and the second respiratory conduit at a time. Alternatively, the second retaining member may be configured to receive the respiratory conduit and the second respiratory conduit simultaneously.

The second ledge may be configured to engage an undulation of the respiratory conduit received within the second interior space and an undulation of the second respiratory conduit received within the third interior space, in use.

The respiratory therapy accessory may include a third retaining member extending from the body, the third retaining member including a hook to retain a further patient interface assembly.

The further patient interface assembly may include a total face mask, a full-face mask, an oro-nasal mask, a nasal mask, a nasal pillows mask, an endotracheal tube or a tracheostomy tube.

The hook may be configured to retain a packaging containing the patient interface assembly.

At least one support member may extend from the body to a respective one of the first retaining member, the second retaining member and the third retaining member. The support member may include a strut or a gusset.

The first retaining member may include a part annular portion that defines the first interior space, and a gusset extending to a proximal end of the part annular portion.

The first retaining member may be spaced apart from the body by a stem, with the gusset extending from a lower surface of the stem.

The second retaining member may include a pair of retaining arms joined to each other at a proximal end, and a gusset extending to the proximal end of the pair of retaining arms.

The third retaining member may include a hook at a distal end of the third retaining member, and a gusset extending to a proximal end of the hook.

In a sixth aspect, a respiratory therapy accessory includes: a body configured to be coupled to a support; and a retaining member extending from the body, the retaining member including a pair of retaining arms joined at a proximal end, a distal end of the pair of retaining arms defining an opening to an interior space between the pair of retaining arms; wherein the pair of retaining arms converge towards one another at a waist region intermediate the proximal end and the distal end, the waist region dividing the interior space into a first retaining space and a second retaining space, the first retaining space configured to receive a first respiratory therapy component and the second retaining space configured to receive a second respiratory therapy component.

The first retaining space and the second retaining space may be configured to retain only one of the respective first respiratory therapy component and the second respiratory therapy component at a time. Alternatively, the first retaining space and the second retaining space may be configured to retain the respective first respiratory therapy component and the second respiratory therapy component simultaneously.

A width of the first retaining space of the interior space may be less than a width of the second retaining space of the interior space.

The pair of retaining arms together may have a substantial figure-eight shape.

The first retaining space of the interior space and the second retaining space of the interior space may each be substantially circular, and a diameter of the first retaining space of the interior space may be less than a diameter of the second retaining space of the interior space.

The distal ends of the pair of retaining arms may diverge outwardly away from each other.

The pair of retaining arms may be spaced from the body by a stem.

At least one of the first respiratory therapy component and the second respiratory therapy component may include a respiratory conduit.

The retaining member may include a ledge extending from the pair of retaining arms towards the interior space, the ledge configured to engage an outer surface of at least one of the first respiratory therapy component or the second respiratory therapy component.

The first respiratory therapy component may include a first respiratory conduit, the second respiratory therapy component may include a second respiratory conduit, and the retaining member may be configured to engage an undulation in the outer surface of at least one of the first respiratory conduit or the second respiratory conduit. The undulation may include a corrugation.

The ledge may be located intermediate a top surface and a bottom surface of the retaining member. The ledge may be located halfway between the top surface and the bottom surface of the retaining member.

A support member may extend from the retaining member and join with the body. The support member may include a strut or a gusset.

The gusset may terminate at the proximal end of the pair of retaining arms.

The pair of retaining arms may be spaced from the body by a stem, and the gusset may extend from a lower surface of the stem.

In a seventh aspect, a respiratory therapy accessory retains a patient interface assembly, the patient interface assembly including a tubing, a patient interface fluidly coupled with one end of the tubing, and a connector attached to an opposing end of the tubing. The respiratory therapy accessory includes a body configured to be coupled to a support, and a retaining member extending from the body, the retaining member including: a part annular portion defining an interior space, the interior space including a connector portion with a first diameter configured to receive the connector, and a tubing portion with a second diameter configured to receive the tubing, the first diameter being greater than the second diameter, and a ledge extending from the part annular portion towards the interior space, the ledge defining the tubing portion, wherein the ledge may be configured to, in use, abut a shoulder of the connector such that the connector rests on the ledge, and the tubing and the patient interface hang from the connector.

The part annular portion may include an arcuate wall defining the connector portion, the ledge and the arcuate wall in combination being configured to cradle the connector.

The ledge may form a bottom surface of the part annular portion.

The first diameter may be equal to or greater than a corresponding outer diameter of the connector.

The second diameter may be equal to or greater than a maximum outer diameter of the tubing.

The part annular portion may be spaced from the body by a stem.

The retaining member may extend in a direction perpendicular to a longitudinal axis of the body.

A distal end of the part annular portion may define an opening to the interior space, a width of the opening being less than an outer diameter of the connector.

The width of the opening may be less than or equal to a maximum outer diameter of the tubing.

Distal ends of the part annular portion may diverge outwardly.

The patient interface assembly may include a non-sealing nasal cannula assembly.

A support member may extend between the retaining member and the body. The support member may include a strut or a gusset.

The gusset may be joined to the part annular portion.

The part annular portion may be spaced from the body by a stem, and the gusset may extend from a lower surface of the stem.

In an eighth aspect, a respiratory therapy accessory includes: a body configured to be coupled to a support, the body including a first arm and a second arm; a slot disposed between the first arm and the second arm, the slot configured to receive the support; and a retaining member extending from the body and configured to retain a respiratory therapy component in use, wherein the first arm and second arm are configured to engage the support when the body is rotated about a rotation axis in a first direction, and wherein the retaining member extends from the body in a direction that is parallel to the rotation axis or in a direction which produces a torque on the body in the first direction.

The retaining member may be diametrically opposed to the slot.

The first arm and the second arm may be part annular.

The body may include an upper opening and a diametrically opposed lower opening, the slot may extend between and connects the upper opening and the lower opening, and the retaining member may be circumferentially equidistant from the upper opening and the lower opening.

The respiratory therapy accessory may include a further retaining member diametrically opposed to the lower opening.

The respiratory therapy accessory may include a further retaining member, and the further retaining member may extend from the body in the other of the direction that is parallel to the rotational axis or in the direction which produces the torque on the body in the first direction.

The retaining member may be a third retaining member and the respiratory therapy accessory may include a first retaining member and a second retaining member, wherein the first retaining member and the second retaining member extend from the body at an acute angle to the third retaining member, when viewed from above.

The first retaining member and the second retaining member may be diametrically opposed to each other, when viewed from above.

The third retaining member may include a hook or part annular structure, the hook or part annular structure configured to hang the respiratory therapy component.

The respiratory therapy component may be one or more of a non-sealing nasal cannula assembly, a respiratory conduit, or a non-invasive patient interface assembly.

The non-invasive patient interface assembly may include a total face mask, a full-face mask, an oro-nasal mask, a nasal mask, or a nasal pillows mask.

The first arm may extend in a circumferential direction from one side of a spine of the body, and the second arm may extend in an opposing circumferential direction from an opposing side of the spine of the body.

The retaining member may extend radially from the spine of the body.

In a ninth aspect, a respiratory therapy accessory includes: a hollow cylindrical body configured to be coupled to a support, the body including a first arm and a second arm, the first arm defining a first opening to an interior space of the body and the second arm defining a second opening to the interior space, and a slot disposed between the first arm and the second arm, the slot configured to partially receive the support into the interior space; and a retaining member extending outwardly from the body and configured to receive and retain a respiratory therapy component in use, wherein the first arm and the second arm are configured to resiliently deflect to further receive the support within the interior space when the body is rotated about a rotation axis in a first direction, and wherein the rotation axis is perpendicular to the retaining member.

The retaining member, the first opening and the second opening may be aligned on a plane.

The retaining member may include a hook.

A support member may support the retaining member. The support member may include a strut or a gusset.

In a tenth aspect, a respiratory therapy accessory includes: a body configured to be coupled to a support, the body including a longitudinal axis; a plurality of elongate arms extending from the body and diverging from each other, wherein adjacent pairs of the plurality of elongate arms each define an interior space configured to receive and retain at least one respiratory therapy component.

The plurality of elongate arms may span an angle of no more than about 180°, preferably no more than about 120° and more preferably no more than about 90°.

Proximal ends of at least one adjacent pair of the plurality of elongate arms may define a part annular portion configured to receive and retain the respiratory therapy component.

The part annular portion may include a part annular ledge, the part annular ledge configured to abut a connector of the respiratory therapy component, in use.

The part annular ledge may be spaced from a top surface of the part annular portion, such that an arcuate wall is defined between the part annular ledge and the top surface of the part annular portion, the arcuate wall configured to partially surround the connector, in use.

The part annular ledge and the arcuate wall in combination may be configured to cradle the connector.

The respiratory therapy component may include a non-sealing nasal cannula assembly, the nasal cannula assembly including a tubing, a nasal cannula fluidly coupled to one end of the tubing, and a connector attached to an opposing end of the tubing, and the part annular portion may be configured to, in use, cradle the connector with the tubing and the nasal cannula hanging therefrom.

One or more of the plurality of elongate arms may include a linear ledge extending towards the interior space, the at least one respiratory therapy component including a respiratory conduit, and the linear ledge may be configured to engage an undulation of the respiratory conduit. The undulation may include a corrugation of the respiratory conduit.

One or more of the plurality of elongate arms may include a protrusion extending towards the interior space, the protrusion intersecting with the linear ledge and configured to retain the respiratory conduit within a defined region of the interior space.

A distal end of at least one of the plurality of elongate arms may include a hook, the hook configured to retain a patient interface assembly. The hook may include a recessed region for locating the patient interface assembly.

Divergence angles between two or more adjacent pairs of the plurality of elongate arms may be equal.

In an eleventh aspect, a respiratory therapy accessory includes: an open-ended hollow body configured to engage a support; and at least one retaining member extending from the body and configured to receive and retain a respiratory therapy component in use, wherein the body, when engaged with the support in use, may be configured to be selectively slid along at least a portion of a length of the support.

The body may be cylindrical and configured to be selectively rotated about a longitudinal axis of the support.

The body may include a resiliently deformable first arm, and a resiliently deformable second arm, wherein the first arm and the second arm at least in part define a continuous angular split in the body, extending from an upper end portion of the body to a diametrically opposed lower end portion of the body.

The first arm and the second arm may extend in opposing circumferential directions.

The first arm may be spaced from the second arm in an axial direction of the body.

The first arm and the second arm may be configured to resiliently deform to receive the support through the continuous angular split upon rotational engagement of the body with the support.

The first arm and the second arm may be configured to partially elastically recover as the support is received inside the hollow body.

The respiratory therapy accessory may include two retaining members extending from the second arm.

In some examples, there may be no retaining member extending from the first arm. In other examples, a first retaining member may extend from the first arm and a second retaining member may extend from the second arm.

A support member may extend between the body and the retaining member. The support member may include a strut or a gusset.

In a twelfth aspect, a respiratory therapy accessory includes: a hollow body including a resiliently deformable first arm, and a resiliently deformable second arm, wherein the first arm and the second arm at least in part define a continuous angular split in the hollow body, extending from a first side at a first end of the hollow body to an opposing second side at an opposing second end of the hollow body; and a retaining member extending from the hollow body and configured to receive and retain a respiratory therapy component in use.

The hollow body may be cylindrical, and the first arm and the second arm may extend in opposing circumferential directions.

The first arm may be spaced from the second arm in an axial direction of the hollow body.

The retaining member may be diametrically opposed to the continuous angular split.

The retaining member may extend from the first side of the hollow body.

The retaining member may extend outwardly from the second end of the hollow body.

The retaining member may include a hook.

The retaining member may include a third retaining member and the respiratory therapy accessory may further include a first retaining member and a second retaining member extending from the hollow body and each configured to receive and retain a respiratory therapy component in use.

The first retaining member and the second retaining member may each form an acute angle with the third retaining member.

The first retaining member, the second retaining member and the third retaining member may span an angle of less than 180°, between about 120° and 160°, or about 140°, when viewed along a longitudinal axis of the body. Alternatively, the first retaining member and the second retaining member may extend from the body in opposing directions when viewed along a longitudinal axis of the body.

The first retaining member, the second retaining member and the third retaining member may each be structurally dissimilar from one another.

The first retaining member, the second retaining member and the third retaining member may each be configured to retain a different respiratory therapy component.

A support member may extend between the hollow body and the retaining member. The support member may include a strut or a gusset.

In a thirteenth aspect, a method for retaining a respiratory therapy component to a support includes steps of: providing a respiratory therapy accessory according to the twelfth aspect, translating the respiratory therapy accessory towards the support until the support is partially received within the hollow body through a central portion of the continuous angular split, rotating the respiratory therapy accessory to engage the first arm and the second arm with the support until the support is further received within the hollow body, and hanging the respiratory therapy component from the first retaining member.

In a fourteenth aspect, a respiratory therapy accessory includes a first retaining member configured to retain a nasal cannula assembly, a second retaining member configured to retain a respiratory therapy component, and a third retaining member configured to retain a respiratory mask.

The respiratory therapy component may include one or more of an inspiratory conduit, an expiratory conduit and a wye-piece.

The first retaining member may be configured to receive and retain a connector of the nasal cannula assembly.

The third retaining member may be configured to be received within an aperture in a packaging containing the respiratory mask.

At least one of the first retaining member, the second retaining member and the third retaining member may be structurally dissimilar from another of the first retaining member, the second retaining member and the third retaining member.

The first retaining member, the second retaining member and the third retaining member may each be structurally dissimilar from one other.

The respiratory therapy accessory may include a body configured to be secured to a support, in use, wherein the first retaining member, the second retaining member and the third retaining member each extend outwardly from the body in different directions.

The body may be configured to be secured to the support by rotation of the respiratory therapy accessory as a whole.

At least one of the first retaining member, the second retaining member and the third retaining member may be supported by a support member. The support member may include a strut or a gusset.

The nasal cannula assembly may include a non-sealing nasal cannula.

The respiratory mask may include any one of a total face mask, a full-face mask, an oro-nasal mask, a nasal mask, or a nasal pillows mask.

In a fifteenth aspect, a respiratory therapy system includes the respiratory therapy accessory of any one of the first, third, fifth to twelfth, or fourteenth aspects, and the support.

The respiratory therapy system may include a humidifier.

The respiratory therapy system may include a gases source.

The respiratory therapy system may include a humidifier and a gases source integrated in a single device.

The gases source may be changed between a mode suitable for nasal high flow therapy and a mode suitable for non-invasive therapy.

The gases source may be configured to operate in mode suitable for invasive ventilation therapy.

The support may be, or include, a pole.

The pole may form at least part of any one or more of an intravenous (IV) bag support, a water bag pole, a roll stand, a ventilator trolley or a bed frame.

The respiratory therapy system may include a nasal cannula assembly, wherein the respiratory therapy accessory is configured to retain the nasal cannula assembly.

In a sixteenth aspect, a respiratory therapy system includes the respiratory therapy accessory of any one of the first, third, fifth to twelfth, or fourteenth aspects, and a nasal cannula assembly, wherein the respiratory therapy accessory is configured to retain the nasal cannula assembly.

The nasal cannula assembly may include a nasal cannula, a tubing fluidly coupled with the nasal cannula at one end, and a connector attached to an opposing end of the tubing and configured to connect the tubing to an inspiratory conduit when the nasal cannula assembly is used for delivery of respiratory therapy to a patient.

The nasal cannula assembly may be a non-sealing nasal cannula assembly.

The respiratory therapy system may include any one or more of a gases source, a humidifier, an integrated gases source and humidifier, the support, an inspiratory conduit, an expiratory conduit, and a wye-piece.

In further aspects, a respiratory therapy accessory may be substantially as described and illustrated herein with reference to any one of the first example, second example, third example, fourth example, or sixth example.

In further aspects, a respiratory therapy accessory may be substantially as shown in any of FIG. 1 to FIG. 12, FIG. 13, FIG. 14, FIG. 15 to FIG. 20C, FIG. 21 or FIG. 22.

Yet further aspects, novel features and advantages of the present disclosure will be readily apparent to those skilled in the art in light any one or more of the illustrative examples set out in the detailed description and drawings. The description and drawings are to be regarded as illustrative in nature, and not restrictive. Modifications or improvements may be made without departing from the spirit or scope of the disclosure and claims.

### BRIEF DESCRIPTION OF THE DRAWINGS

A number of non-limiting examples will be described with reference to the accompanying drawings. To easily identify the discussion of any particular element, the most significant digit or digits in a reference numeral refers to the figure number in which that element is first indicated.

FIG. 1 illustrates a perspective view of a respiratory therapy accessory in accordance with a first example.

FIG. 2 illustrates a further perspective view of the first example of the respiratory therapy accessory of FIG. 1.

FIG. 3 illustrates a plan view of the respiratory therapy accessory of FIG. 1.

FIG. 4 illustrates a side view of the respiratory therapy accessory of FIG. 1.

FIG. 5 illustrates a rear view of the respiratory therapy accessory of FIG. 1.

FIG. 6A, FIG. 6B, FIG. 6C and FIG. 6D illustrate steps for attaching the respiratory therapy accessory of FIG. 1 to a support, in use.

FIG. 7 illustrates a detailed perspective view of a retaining member of the respiratory therapy accessory of FIG. 1.

FIG. 8 illustrates a sectioned detailed perspective view of the respiratory therapy accessory of FIG. 1, retaining a respiratory therapy component in use.

FIG. 9 illustrates a detailed perspective view of another retaining member of the respiratory therapy accessory of FIG. 1.

FIG. 10 illustrates a sectioned detailed perspective view of the respiratory therapy accessory of FIG. 1 retaining another respiratory therapy component.

FIG. 11 illustrates a perspective view of the respiratory therapy accessory of FIG. 1 attached to a support and retaining two respiratory therapy components, in use.

FIG. 12 illustrates a perspective view of the respiratory therapy accessory of FIG. 1 attached to a support adjacent a sterile water bag, in use.

FIG. 13 illustrates a perspective view of a respiratory therapy accessory in accordance with a second example.

FIG. 14 illustrates a bottom perspective view of a respiratory therapy accessory in accordance with a third example.

FIG. 15 illustrates a perspective view of a respiratory therapy accessory in accordance with a fourth example.

FIG. 16 illustrates a side view of the respiratory therapy accessory of FIG. 15.

FIG. 17 illustrates a rear view of the respiratory therapy accessory of FIG. 15.

FIG. 18 illustrates a bottom view of the respiratory therapy accessory of FIG. 15.

FIG. 19 illustrates a front view of the respiratory therapy accessory of FIG. 15.

FIG. 20A, FIG. 20B and FIG. 20C illustrate steps for attaching the respiratory therapy accessory of FIG. 15 to a support, in use.

FIG. 21 illustrates a perspective view of a respiratory therapy accessory in accordance with a fifth example.

FIG. 22 illustrates a perspective view of a respiratory therapy accessory in accordance with a sixth example.

### DETAILED DESCRIPTION

Nasal high flow (NHF) therapy is a form of high flow therapy (HFT) delivered to a patient by way of a non-sealing nasal cannula assembly.

High flow therapy as discussed herein is intended to be given its typical ordinary meaning, as understood by a person of skill in the art, which generally refers to a respiratory assistance system delivering a targeted flow of humidified respiratory gases via an intentionally unsealed patient interface assembly with flow rates generally intended to meet or exceed inspiratory flow of a patient. Typical patient interface assemblies include, but are not limited to, a nasal or tracheal patient interface assembly. Typical flow rates for adult patients often range from, but are not limited to, about fifteen liters per minute to about sixty liters per minute or greater. Typical flow rates for pediatric patients (such as neonates, infants and children) often range from, but are not limited to, about one liter per minute per kilogram of patient weight to about three liters per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names.

For example, in some configurations, for an adult patient ‘high flow therapy’ may refer to the delivery of gases to a patient at a flow rate of greater than or equal to about 10 liters per minute (10 liters per minute (LPM)), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with a neonatal, infant, child or adult patient may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing the work of breathing. Additionally, high flow therapy may generate a flushing effect in the nasopharynx such that the anatomical dead space of the upper airways is flushed by the high incoming gases flow. The flushing effect can create a reservoir of fresh gas available for each and every breath, while minimizing re-breathing of carbon dioxide, nitrogen, etc.

The patient interface assembly for use in high flow therapy can be a non-sealing patient interface assembly to prevent barotrauma, which can include tissue damage to the lungs or other organs of the patient's respiratory system due to difference in pressure relative to the atmosphere. The patient interface assembly can be a nasal cannula assembly with a manifold and nasal prongs, and/or a face mask, and/or a nasal pillows mask, and/or a nasal mask, and/or a tracheostomy interface assembly, or any other suitable type of patient interface assembly.

Unless the context clearly requires otherwise, throughout the description and claims the words “including,” “comprising” and variants are to be construed in an inclusive rather than exclusive sense. That is, the terms should be construed in the sense of “including, but not limited to,” as opposed to “consisting solely of.” Similarly, any examples listed in the following description are not intended to be limiting.

The phrases “when viewed along the longitudinal axis,” “when viewed from above,” and the like refer to the relative arrangement of components as apparent from that particular perspective only. For example, where two components which are said to be diametrically opposed when viewed along the longitudinal axis, that description is not intended to require (nor exclude) those same components being spaced along the longitudinal axis (i.e., when viewed perpendicular to the longitudinal axis). Similarly, when two components are said to define an angle therebetween when viewed along the longitudinal axis (of from above/below), that description is not intended to require (nor exclude) those same components not being spaced from each other along longitudinal axis.

Any reference to publications or products throughout this specification, including the background, should in no way be considered as an admission that the publication or product is prior art, analogous, widely known or forms part of common general knowledge in the field.

### First Example

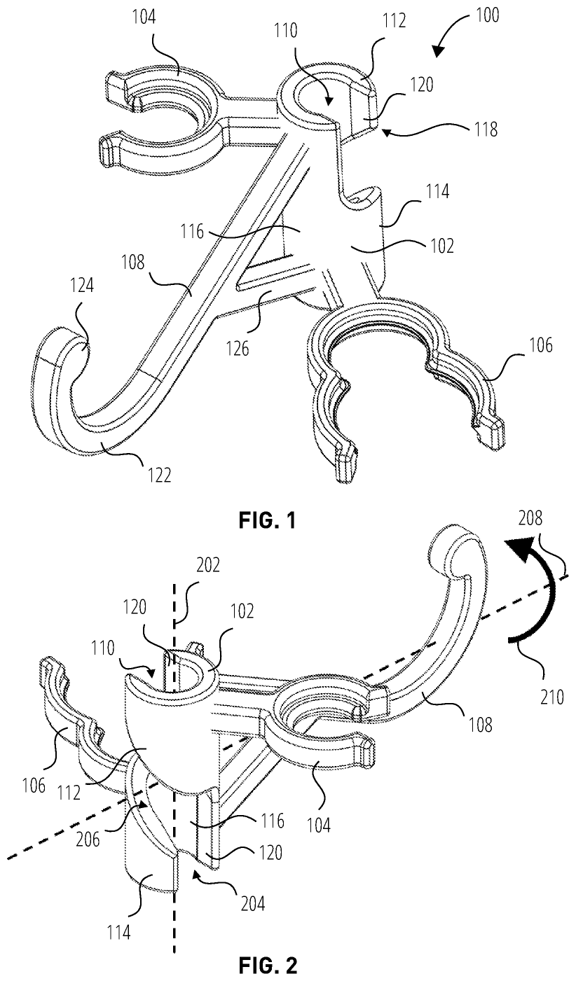

Referring first to FIG. 1 to FIG. 12 there is shown a respiratory therapy accessory 100 according to a first example. The respiratory therapy accessory 100 includes a body 102 configured to be attached to a support 602, and three retaining members 104, 106, 108 each configured to retain a respiratory therapy component.

### Body

The body 102 of respiratory therapy accessory 100 is a generally cylindrical hollow open-ended structure defining an elongate interior space 110. The body 102 is open to the interior space 110 at each of its opposing longitudinal ends. The body 102 includes a first arm 112 and a second arm 114 axially spaced from each other. The elongate body 102 and/or the axial spacing of the arms 112, 114 may improve stability of the respiratory therapy accessory 100 when attached to the support.

The first arm 112 and second arm 114 are each part annular and extend arcuately about a longitudinal axis 202 of the body 102 in opposing circumferential directions, cantilevered from a spine 116 formed by, in this example, a front portion of the body 102. The spine 116 of the body 102 of this first example extends continuously between the opposing longitudinal ends of the body 102 in a direction parallel to the longitudinal axis 202.

The body 102 of the respiratory therapy accessory 100 is formed, at least in part, from a resiliently deformable material, such as a resilient plastics material, so that the first arm 112 and second arm 114 are configured to resiliently deform in use upon attachment of the body 102 to a support, as described in further detail below. In some examples, the respiratory therapy accessory 100 may be formed from a polymer, such as a rigid thermoplastic polymer that has sufficient creep resistance, strength, and other such material properties. Suitable materials include acetal, nylon, or polyketone, for example.

The retaining members 104, 106, 108 may be integrally molded with the body 102. The respiratory therapy accessory 100 may be left attached to a support for extended periods and may be used with more than one patient. The respiratory therapy accessory 100 may be periodically sterilized by wiping down with a disinfectant. In some examples, the material may be selected so that the respiratory therapy accessory 100 is autoclavable.

As best shown in FIG. 1 and FIG. 2, the first arm 112 defines a first portion of the interior space 110. The first arm 112 extends arcuately from a first side of the spine 116, defining a first opening 118 to the interior space 110 between a distal end of the first arm 112 and an opposing second side of the spine 116.

As best shown in FIG. 2 and FIG. 4, the second arm 114 defines a second portion of the interior space 110. The second arm 114 extends arcuately from the second side of the spine 116, defining a second opening 204 between the distal end of the second arm 114 and the first side of the spine 116.

The first opening 118 and the second opening 204 are disposed on opposing sides and opposing ends of the body 102.

The first opening 118 of respiratory therapy accessory 100 has a width that is narrower than a diameter of the first portion of the interior space 110 (that is, the internal diameter of the body 102). The second opening 204 similarly has a width that is less than a diameter of the second portion of the interior space 110.

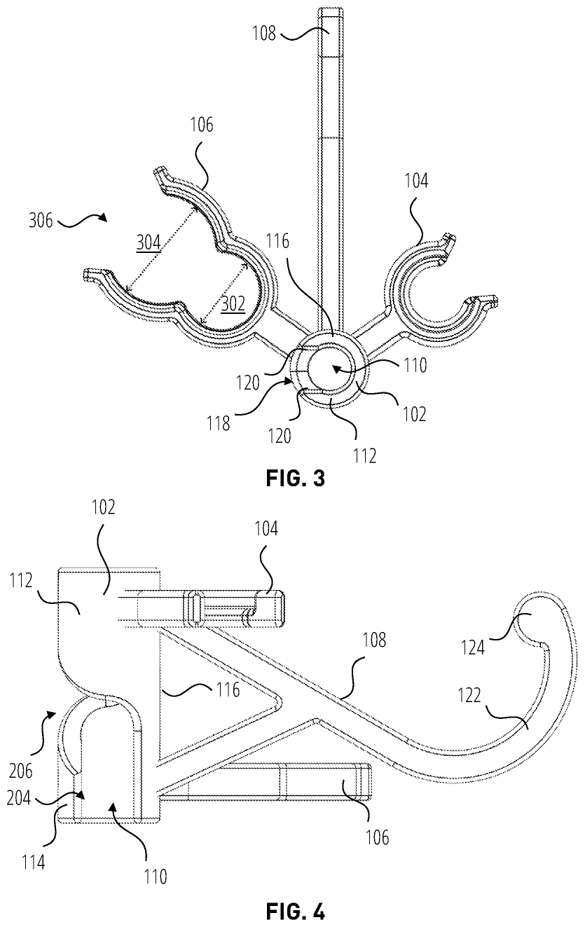

The distal ends of the first arm 112 and the second arm 114, and the opposing portions of the spine 116 include substantially planar opposing edges 120 at the respective first opening 118 and second opening 204. In respiratory therapy accessory 100 the opposing edges 120 extend substantially parallel with longitudinal axis 202. The opposing edges 120 may be substantially parallel with each other. In other examples, the opposing edges may diverge outwardly and/or a pair of outwardly diverging extensions may be provided at the distal ends of the arms, similar to the first retaining member 104 and second retaining member 106 as best shown in FIG. 3. These variations may each assist in guiding a support into, and through, the respective openings when the respiratory therapy accessory 100 is attached to a support as discussed in further detail below.

The opposing edges 120 are configured to determine, in part, a torque required to engage the first arm 112 and the second arm 114 of the body 102 with a support, as described in further detail below, such that the support is received within the interior space 110. Other factors that can influence the torque required to rotate the body 102 onto the support include, but are not limited to, the material of the body 102 and/or the support, the shape of the interior space 110, and the shape of the first arm 112 and/or second arm 114, for example.

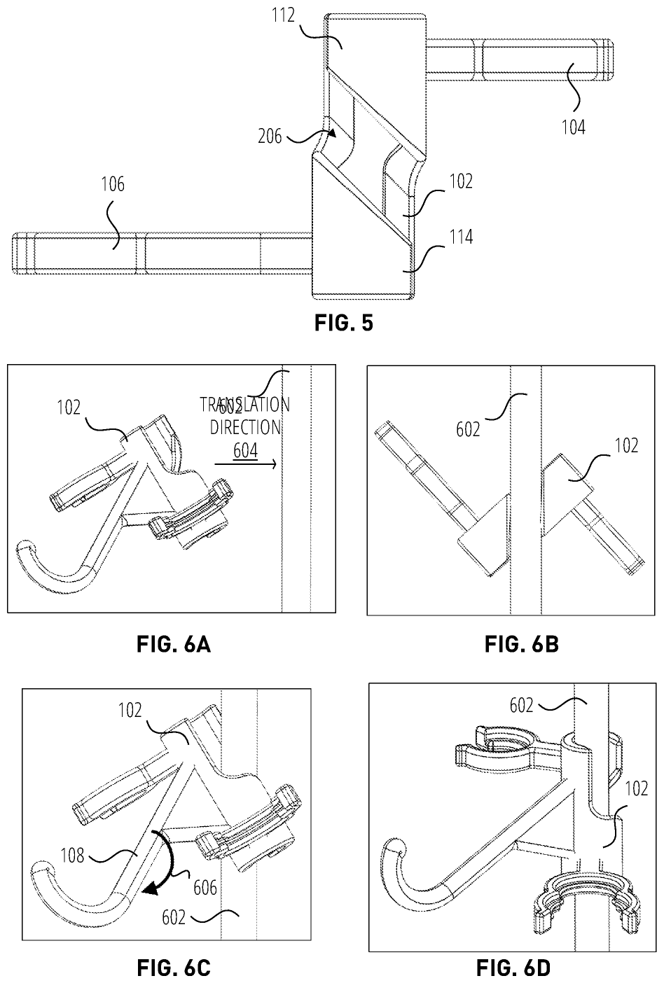

Referring in particular to FIG. 2 and FIG. 5, a slot 206 extends between, and connects, the first opening 118 and the second opening 204. The slot 206 is configured to initially receive the support partially into the interior space 110 upon attachment of the respiratory therapy accessory 100, as described in further detail below. The slot 206 of respiratory therapy accessory 100 is angled with respect to the longitudinal axis 202 of the body 102 when viewed from the rear, as shown in FIG. 5. An angled slot 206 may advantageously indicate to the user the direction of rotation to engage and/or disengage the respiratory therapy accessory 100 with the support. In other examples, the slot 206 may be perpendicular to the longitudinal axis 202 of the body 102.

The first opening 118, second opening 204 and slot 206 in combination can be seen to define a continuous angular split in the hollow cylindrical body 102. The continuous angular split extends from one side of the body 102 at a first end to an opposing second side at an opposing second end of the body 102. At opposing ends of the body, the split extends substantially parallel to the longitudinal axis 202. A central portion of the split extends, at least in part, circumferentially from one side of the body to the other.

The slot 206 of respiratory therapy accessory 100 may be slightly wider than the diameter of the support which the respiratory therapy accessory 100 is intended to attach to. In other examples, however, the slot 206 may, at least in part, be equal to or narrower in width than the diameter of the support. In such examples, at least one of the body 102 and/or support may be configured to deform as the support is received by the slot 206.

As best shown in FIG. 5, a lower edge of the first arm 112 and an upper edge of the second arm 114 may be beveled or chamfered to help guide the slot 206 about a support, correcting any minor misalignment.

### Attachment of the Body to a Support

The respiratory therapy accessory 100 has no moving parts such as hinges, screws or clamp jaws. It is configured to clip onto to a support by merely translating the respiratory therapy accessory 100 towards the support then rotating the respiratory therapy accessory 100 as a whole to engage the support. This may advantageously make the respiratory therapy accessory 100 inexpensive to manufacture and/or simple and intuitive to use. It may advantageously enable the respiratory therapy accessory 100 to be selectively engaged and/or disengaged anywhere along the length of the support. In some examples, the respiratory therapy accessory 100 may alternatively be slid down directly onto and over the top end of the support, in the axial direction. The open ends of the body 102 may be beveled or chamfered to facilitate this.

Attachment of respiratory therapy accessory 100 to a support is described in further detail with particular reference to FIG. 6A to FIG. 6D.

In FIG. 6A the respiratory therapy accessory 100 is shown adjacent a support 602. The respiratory therapy accessory 100 of this first example is configured to be attached to a support 602 in the form of a tubular steel pole having a circular cross-section, such as a pole forming part of an intravenous (IV) bag support, water bag pole, roll stand, ventilator trolley or bed frame, by way of non-limiting example. The first arm 112 and second arm 114 define an interior space 110 having a shape that generally corresponds to the outer surface of the support 602. As shown in FIG. 2, the first arm 112 and second arm 114 define an annular shape suitable for engaging the cylindrical shaft of the pole, and may also be suitable for engaging certain other supports having cross-sections which approximate a circle, such as a hexagonal cross-section. In other examples, however, a respiratory therapy accessory according to the present disclosure may be configured for attachment to other supports, including supports having rectangular, square, elliptical, or polygonal cross-sections, by way of non-limiting example, by having a correspondingly-shaped interior space 110.

To attach the body 102 to the support 602, the body 102 may be rotated to align the slot 206 generally parallel with the support 602, as best shown in FIG. 6B, and moved towards the support 602 in the direction indicated by arrow translation direction 604 in FIG. 6A so that the support 602 is received by the slot 206.

In FIG. 6B, the support 602 is shown partially received within the interior space 110, having passed through the slot 206. The slot 206 at this stage remains aligned with the support 602.

As described above, the slot 206 is slightly wider than the diameter of the support 602. In alternative examples, however, the width of the slot 206 may be slightly narrower than the diameter of the support 602, and the body 102 and/or the support 602 may be configured to deform as the support 602 passes through the slot 206 and partially into the interior space 110. This may ameliorate or prevent inadvertent disengagement of the body 102 from the support 602.

To engage the first arm 112 and second arm 114 with the support 602 to securely attach the body 102 to the support 602, the body 102 may be rotated in an anti-clockwise direction as illustrated in FIG. 6B (or a clockwise direction when viewed from the front of the respiratory therapy accessory 100).

Referring again to FIG. 2, the rotation is about a rotation axis 208. The rotation axis 208 for respiratory therapy accessory 100 is perpendicular to the longitudinal axis 202 and passes through slot 206 and spine 116 of the body 102. For attaching the body 102 to the support 602, rotation is in the direction indicated by arrow 210. To disengage the first arm 112 and second arm 114, the body may be rotated about rotation axis 208 in the opposite direction.

Referring to FIG. 6C, rotation may be effected by grasping and rotating the third retaining member 108 as indicated by the arrow 606.

As the body 102 is initially rotated, the support 602 is received by the first opening 118 defined by the first arm 112 at one end of the body 102 and the second opening 204 defined by the second arm 114 at the opposing end of the body 102. As the body 102 is rotated further, the first arm 112 and second arm 114 are caused to resiliently deform outwardly to accommodate the support 602 within the respective openings 118, 204, between the opposing edges 120, then elastically recover as the opposing edges 120 pass the widest portion of the support 602, and the support 602 is further received within the interior space 110. In some examples, sudden elastic recovery of at least one of the first arm 112 and second arm 114 may urge the respiratory therapy accessory 100 into final engagement with the support and provide tactile and/or audible feedback confirming secure attachment of the body 102 to the support 602 with a “snap.” That is, the body 102 can be secured to the support with a snap-fit by rotation of the respiratory therapy accessory 100 as a whole.

FIG. 6D illustrates the body 102 attached to the support 602, with support 602 received fully within the interior space 110.

The body 102 of the respiratory therapy accessory 100 may be formed to have an interior space 110 with a diameter which is slightly less than the width of the support 602, prior to attachment. That is, at least one of the first arm 112 and second arm 114 may be configured so that it does not fully elastically recover when the support 602 is further received, for example fully received, within the interior space 110. The resilience of first arm 112 and second arm 114 may therefore apply a compressive force to the support 602. This may increase friction between the body 102 and support 602 to ameliorate or avoid the respiratory therapy accessory 100 inadvertently slipping down the support 602. Alternatively, or additionally, at least part of the body 102 may be formed from, or lined with, a slip-resistant material having a relatively higher coefficient of friction with the pole than one or more of nylon, acetal or polyketone. By way of non-limiting example, at least part of an interior surface of a nylon body 102 may be overmolded with an elastomeric material such as liquid silicone rubber (LSR).

The body 102 of respiratory therapy accessory 100 may be configured to selectively slide along the support 602, and/or rotate about the longitudinal axis of the support 602, when a sufficient external force is applied to the body 102, such as by a user's hand, to position the respiratory therapy accessory 100 at a desired location (e.g., height) and orientation on the support 602. The respiratory therapy accessory 100 is configured such that a force required to slide the respiratory therapy accessory 100 along the support 602 exceeds the force due to gravity acting upon the combination of the respiratory therapy accessory 100 and any respiratory therapy components which may be expected to be retained by the respiratory therapy accessory 100 simultaneously, yet not so great that the respiratory therapy accessory 100 cannot be selectively moved with relative ease by a user. In some examples, the respiratory therapy accessory 100 may be provided with a momentary actuator, such as a pair of levers attached to the first arm 112 and second arm 114 which may be compressed together, to selectively deform at least one of the first arm 112 and second arm 114 outwardly, temporarily reducing friction with the support and thus the force required to slide and/or rotate the body 102.

In some examples, the respiratory therapy accessory 100 may include a locking mechanism (not shown) configured to lock the body 102 to the support 602, thus ameliorating or avoiding the body 102 inadvertently sliding down the support 602. For example, the locking mechanism may include a grub screw or cam that abuts against an external surface of the support 602 to secure the body 102 in position relative to the support 602. To move the body 102 along the support 602, a user can loosen the grub screw or cam, slide the body 102 along the support 602, and then retighten the grub screw or cam once the body 102 is in the desired position.

### Retaining Members

The retaining members may each be configured to hold a respiratory therapy component. The respiratory therapy component may be a respiratory conduit, a patient interface assembly, or packaging for a patient interface assembly, by way of non-limiting example.

The respiratory therapy accessory 100 of the first example includes three retaining members 104, 106, 108 each configured to hold a different respiratory therapy component. More specifically, the first retaining member 104 is configured to retain a first patient interface assembly, the second retaining member 106 is configured to retain a first and/or second respiratory conduit, and the third retaining member 108 is configured to retain a second patient interface assembly. The first and second patient interface assemblies may each be a different type of patient interface. Each of the first retaining member 104, second retaining member 106 and third retaining member 108 may therefore have a different shape whereby they are structurally dissimilar to each other.

In other examples, a respiratory therapy accessory may be provided with one or two of the first retaining member 104, the second retaining member 106 or the third retaining member 108; one or more alternative retaining members; two or more identical or similar retaining members (including, but not limited to, two or more of any one of the retaining members 104, 106, or 108); a combination of similar and dissimilar retaining members; four or more retaining members; or any combination of the foregoing.

Referring back to FIG. 5, the first retaining member 104 of the example respiratory therapy accessory 100 is spaced from the second retaining member 106 along the longitudinal axis 202 of the body 102. As shown in FIG. 1 and FIG. 5, for example, the first retaining member 104 extends from body 102 at or adjacent the first arm 112, towards an upper end of the body 102, and the second retaining member 106 extends from the body 102 at or adjacent the second arm 114, towards a lower end of the body 102. As best shown in FIG. 5, the slot 206 is located between the first retaining member 104 and the second retaining member 106.

In other examples, the first retaining member 104 and the second retaining member 106 may be spaced from one end of the body 102 a similar or identical distance. For example, the first retaining member 104 can extend, for example bifurcate, from the same point of the body 102 which the second retaining member 106 extends from. In such an example, the slot 206 may be located above (or below) both the first retaining member 104 and the second retaining member 106.

Referring again to FIG. 2, the third retaining member 108 extends from the body 102 in a direction that is parallel to, or aligned with, the rotational axis 208, at least when viewed along the longitudinal axis 202 (as shown in FIG. 3, for example). That is, the third retaining member 108 extends from the spine 116, circumferentially equidistant from the first opening 118 and the second opening.

The second retaining member 106 extends from the body 102 in a direction which may produce a torque on the body 102 in the direction of the arrow 210. That is, the second retaining member 106 extends from a side of the body generally the same as that including upper first opening 118, and generally opposing the side including the lower second opening 204. As shown, the second retaining member 106 may extend in a direction which is not perpendicular to the axis 208, when viewed along the longitudinal axis 202, as best shown in FIG. 3. That is, the second retaining member 106 extends in a direction which does not directly oppose the second opening 204 (although, in alternative examples, it may do so as described in relation to the second example, below).

Downward forces on each of the third retaining member 108 and second retaining member 106 in use will therefore tend not to create a torque which would tend to disengage the first arm 112 from the support 602.

The first retaining member 104 extends from the body 102 in a direction that may produce a torque about the rotation axis 208 in the opposite direction to the arrow 210. The first arm 112 and/or the second arm 114 are configured to grip onto the support 602 sufficiently to overcome this torque. The torque may also be ameliorated to some extent by the first retaining member 104 extending in a direction which is not perpendicular to the axis 208, when viewed along the longitudinal axis 202 as best shown in FIG. 3. That is, the first retaining member 104 extends in a direction which does not directly oppose the first opening 118. The torque may also be balanced by an opposing torque produced by the second retaining member 106. In other examples, first retaining member 104 may be omitted, or may extend in a direction directly or generally opposing that of the third retaining member 108.

Referring in particular to FIG. 3, it can be seen that the first retaining member 104 and the second retaining member 106 of respiratory therapy accessory 100 each form an acute angle with the third retaining member 108 when viewed along the longitudinal axis 202. The acute angles in this example are equal. That is, the third retaining member 108 bisects the angle between the first retaining member 104 and the second retaining member 106.

The first retaining member 104 of the example respiratory therapy accessory 100 may be configured to hold a patient interface assembly for use in delivering breathing gases to a patient for nasal high flow (NHF) therapy. The patient interface assembly may be a non-sealing nasal cannula assembly including a nasal cannula, tubing fluidly coupled with the nasal cannula at one end, and a connector attached to an opposing end of the tubing and configured to connect the tubing to an inspiratory conduit when the nasal cannula assembly is used for delivery of respiratory therapy to a patient. The nasal cannula may include a pair of prongs for insertion into the patient's nares, fluidly coupled with a manifold configured to receive respiratory gases from the tubing. Headgear may also be attached to the nasal cannula to secure the nasal cannula assembly to the patient's head when used for delivery of respiratory therapy to a patient.

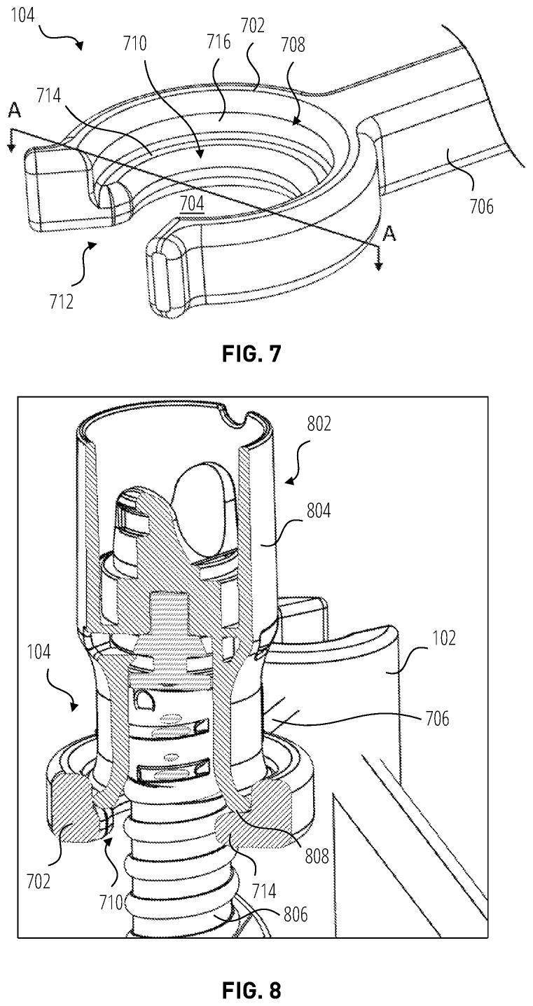

Referring in particular to FIG. 7 and FIG. 8, the depicted first retaining member 104 of respiratory therapy accessory 100 is configured to hold either an Optiflow™+nasal cannula assembly or an Optiflow™ 3S nasal cannula assembly, both available from Fisher & Paykel Healthcare of Auckland, New Zealand. An Optiflow™+nasal cannula assembly 802 is shown by way of example in FIG. 8.

The first retaining member 104 includes a part annular portion 702 defining an interior space 704. The first retaining member 104 also includes a stem 706 that spaces the part annular portion 702 from the body 102 (omitted from FIG. 7). In some examples, the first retaining member 104 may omit stem 706, such that the part annular portion 702 extends directly from the body 102.

The interior space 704 includes an upper connector portion 708 configured to receive a connector 804 of the nasal cannula assembly 802, and a lower tubing portion 710 configured to receive the tubing 806 of the nasal cannula assembly 802. The connector portion 708 and the tubing portion 710 are arranged coaxially. A width of the connector portion 708 is greater than a corresponding width of the tubing portion 710. For example, a diameter of the connector portion 708 is greater than a diameter of the tubing portion 710.

Distal ends of the part annular portion 702 define an opening 712 to the interior space 704. The opening 712 is configured to allow the resilient tubing 806 of the nasal cannula assembly to pass through, but to impede passage of the relatively rigid connector 804. The distal ends of the part annular portion 702 diverge away from the interior space 704, which are thereby configured to guide the tubing 806 towards and through the opening 712 into the interior space 704. The tubing 806 may be resiliently deformable. The tubing may be corrugated. In some examples, a width of the opening 712 may be less than or equal to a maximum outer diameter of the tubing 806. The tubing 806 may deform as it passes through the opening 712 with the application of a suitable insertion force. This can reduce the likelihood of the tubing 806 inadvertently passing back out through the opening 712, which may cause the nasal cannula assembly 802 to become dislodged from the first retaining member 104 and fall to the ground. In some examples, however, the width of the opening 712 may be greater than the external diameter of the tubing 806, such that the tubing 806 need not deform as it passes through the opening 166.

The width of the connector portion 708 of part annular portion 702 is slightly wider than a corresponding width of the connector 804. For example, the connector portion 708 defines a diameter that is marginally wider than an outside diameter of the connector 804 at a proximal end thereof, as shown in FIG. 8.

The tubing portion 710 of the part annular portion 702 is defined in part by a ledge 714. The ledge 714 extends from the part annular portion 702 towards the interior space 704. As best shown in FIG. 8, the ledge 714 gives the part annular portion 702 a substantially “L”-shaped cross-section.

The ledge 714 is configured to abut a lower surface of the connector 804, such that the connector 804 rests upon the ledge 714 substantially coaxial with the part annular portion 702 and interior space 704. The tubing 806 of the nasal cannula assembly 802 is suspended from the connector 804. The nasal cannula, and attached headgear (neither shown), hang from an opposing end of the tubing 806.

In respiratory therapy accessory 100, the ledge 714 forms a bottom surface of the part annular portion 702. In other words, the ledge 714 projects inwardly from an inferior region of the part annular portion 702. This allows for the superior connector portion 708 to accommodate a lower end of the connector 804, as described in further detail below. In alternative examples, however, the ledge 714 may project inwardly from the superior or intermediate regions of the part annular portion 702. For example, the ledge 714 may alternatively project inwardly into the interior space 704 from a mid-point of the part annular portion 702 (i.e., defining a “T”-shaped cross-section). This may be advantageous if the respiratory therapy accessory is to be configured to be equally useable when attached to a support in either orientation.

An arcuate wall 716 extends between an upper surface of the part annular portion 156 and an upper surface of the ledge 714. The arcuate wall 716 is configured to partially surround, and reduce lateral movement of, the connector 804 within the connector portion 708 of the interior space 704 as shown in FIG. 8. The arcuate wall 716 and upper surface of the ledge 714 in combination define the connector portion 708 which is configured to cradle the lower end of the connector 804.

The part annular portion 702 is configured to partially surround the connector 804 through an angle of more than 180°. That is, the part annular portion 702 is configured to extend around more than half of the circumference of the connector 804 and tubing 806 of the nasal cannula assembly. This can ameliorate or prevent inadvertent dislodgement of connector 804 from the first retaining member 104. Thus, to place the connector 804 on the ledge 714, the user is required to position the connector 804 above the top surface of the part annular portion 702, then lower the connector 804 into the interior space 704 until the connector 804 abuts and rests upon the ledge 714 in the connector portion 708. Similarly, removal of the connector 804 in a transverse direction may inhibited by arcuate wall 716 unless the connector 804 is first elevated above the arcuate wall 716 of the part annular portion 702. This may be advantageous in further inhibiting inadvertent dislodging of the nasal cannula assembly 802 from the first retaining member 104, since the connector 804 may be constructed of a rigid plastic material which will not readily deform to allow the connector 804 to pass through the opening 712. In some examples, however, the part annular portion 702 may be configured to elastically deform sufficiently to permit removal of the connector 804 in a transverse direction, optionally with application of sufficient force. This may be advantageously ameliorate or prevent damage to the part annular portion 702 and/or connector 804.

FIG. 8 shows a sectioned view of FIG. 7 taken at A-A, and additionally illustrates, in part, the nasal cannula assembly 802. The connector 804 of nasal cannula assembly 802 defines a shoulder 808. The shoulder 808 is the lower portion of the illustrated connector 804, which is of greater diameter than the adjacent tubing 806. The shoulder 808 is configured to abut the ledge 714. The shoulder 808 may be configured to be positioned at or adjacent a corner defined between the ledge 714 and the arcuate wall 716. The arcuate wall 716 may be configured to partially surround the shoulder 808. It can be seen in FIG. 8 that the shoulder 808 is cradled by the combination of ledge 714 and arcuate wall 716.

The tubing 806 hangs from the connector 804. The diameter of the tubing 806 may be less than the diameter of the tubing portion 710 of first retaining member 104, such that the tubing 806 hangs freely within the tubing portion 710, suspended from the connector 804. As shown in FIG. 8, the connector 804 in use may be retained by the ledge 714 in a substantially upright position so that the tubing hangs substantially vertically, coaxial with the connector 804 and part annular portion 702. In some examples, however, the support 602 may not necessarily be precisely vertical or the first retaining member 104 may be angled so that it is not precisely horizontal. In such examples, the connector 804 may not necessarily be held precisely upright, and at least part of the tubing may not be precisely vertical or coaxial with the hanging connector 804 in use. Moreover, the weight of the nasal cannula and headgear (not shown) upon the tubing 806 may cause the tubing to hang at an angle to vertical. Hanging the nasal cannula assembly by the connector 804 may be advantageous in facilitating drainage of any condensate present in the tubing 806 following use in delivering humidified breathing gases to a patient.

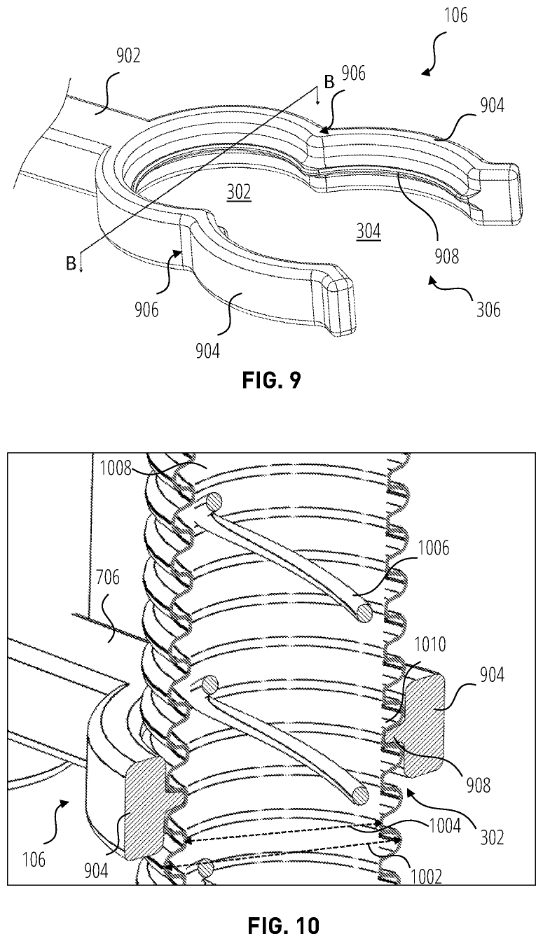

Referring to FIG. 9, the second retaining member 106 is configured to hold a first and/or a second respiratory conduit for use in delivering a flow of breathing gases to a non-sealing nasal cannula assembly for nasal high flow (NHF) therapy, or to a sealing patient interface assembly for non-invasive ventilation (NIV) therapy.

Various respiratory conduits may be corrugated, spirally wound so as to have one or more helical undulations in its outer surface, or may have a smooth, non-undulating outer surface. For example, a respiratory conduit may have a first elongate member spirally wound with a second elongate member to define a lumen and an undulating outer surface. The corrugated and spirally wound respiratory conduits each have a series of alternating peaks and troughs in the axial direction, and therefore may be said to have a maximum outer diameter and a minimum outer diameter. Briefly referring to FIG. 10, the maximum outer diameter 1002 refers to the outside diameter of the conduit when measured peak-to-peak on opposing sides of the conduit. The minimum outer diameter 1004 refers to the outside diameter of the conduit when measured trough-to-trough on opposing sides of the conduit. The difference between the maximum outer diameter and minimum outer diameter will generally be greater for a corrugated conduit than a spirally wound conduit or other conduit having an undulating outer surface. For the purposes of this disclosure, a conduit having a smooth, non-undulating outer surface may also be regarded as having a maximum outer diameter and minimum outer diameter, although it will be appreciated that these may be substantially equal.

The second retaining member 106 can be configured to hold each of a first respiratory conduit and a second respiratory conduit. The first respiratory conduit may have a relatively larger maximum outer diameter and/or minimum outer diameter, and the second respiratory conduit may have a relatively smaller maximum outer diameter and/or minimum outer diameter. The first and second respiratory conduits may be configured for use in different forms of respiratory therapy, e.g., NIV and NHF, respectively. In another example, the second retaining member 106 can be configured to hold each of a first respiratory conduit suitable for use for adults, and a second respiratory conduit of smaller maximum outer diameter suitable for use for infants and/or pediatric patients. In some examples, however, the first and second respiratory conduits may have the same maximum and minimum outer diameters.

The respiratory conduits may include one or more heating elements 1006 configured to heat the gases flowing through the respiratory conduit from a gases source, such as a ventilator, a gas bottle, or a wall source, for example. Some respiratory conduits may also include a patient-end temperature sensor or other sensors. The respiratory conduit can be configured to couple with an outlet of a humidifier at one end, and a patient interface assembly at an opposing end. The humidifier can be located between a ventilator and a patient, or can be integrated with a blower. The respiratory conduit can be part of a dual limb circuit. That is, the respiratory conduit, in the form of an inspiratory conduit, can be coupled via a wye-piece with an expiratory conduit that delivers exhaled gases from the patient back to a ventilator. The second retaining member 106 can be configured to directly retain the inspiratory conduit, thereby indirectly retaining the expiratory conduit and wye-piece. Alternatively, the second retaining member 106 may directly retain the expiratory conduit. In some examples, the second retaining member 106 may be configured to directly retain both the inspiratory conduit and expiratory conduits simultaneously.

FIG. 9 illustrates the example second retaining member 106 in detail, with the body 102 and other retaining members of the respiratory therapy accessory 100 omitted for clarity. The second retaining member 106 includes a stem 902 and a pair of retaining arms 904. The pair of retaining arms 904 are spaced apart from the body 102 by the stem 902. The stem 902 can space one or more respiratory therapy components retained by the second retaining member 106 from the body 102 and support 602. The stem 902 increases the distance of the second retaining member 106 from the body 102 which in turn increases the torque acting on the support 602, when in use, which may further stabilize the body 102 on the support 602. In some examples, however, the stem 902 may be omitted.

The pair of retaining arms 904 are joined at a proximal end at or adjacent the stem 902. Distal ends of the pair of retaining arms 904 define an opening 306 to the interior space. The pair of retaining arms 904 converge towards one another at a waist region 906 intermediate the respective distal and proximal ends, such that the pair of retaining arms 904 define a first retaining space 302 and a second retaining space 304. The first retaining space 302 and the second retaining space 304 together define a single continuous interior space. That is, the waist region 906 notionally divides the single continuous interior space defined by the retaining arms 904 into a first retaining space 302 and a second retaining space 304 which are each substantially circular. The pair of retaining arms 904 and the combination of the first retaining space 302 and the second retaining space 304 therefore each generally have a generally figure-eight shape. The waist region 906 is between the first retaining space 302 and the second retaining space 304, and is narrowed with respect to the adjacent first retaining space 302 and second retaining space 304. The first retaining space 302 is located closer towards the body 102 than the second retaining space 304. The opening 306 provides direct entry to the second retaining space 304, and indirect entry to the first retaining space 302 via the second retaining space 304.

The distal ends of the pair of retaining arms 904 diverge outwardly away from the second retaining space 304. The divergent distal ends are configured to guide a respiratory conduit towards and through the opening 306, into the second retaining space 304. The pair of retaining arms 904 and/or the respiratory conduit may resiliently deform as the respiratory conduit passes through the opening 306.

The first retaining space 302 and the second retaining space 304 of the example respiratory therapy accessory 100 are each configured to receive a different respiratory conduit. As best shown in FIG. 3, it will be observed that the first retaining space 302 and the second retaining space 304 each have a different width or diameter, with the relatively larger second retaining space 304 being located adjacent the opening 306. The second retaining space 304 is therefore configured to retain a respiratory conduit having a relatively larger maximum outer diameter and/or minimum outer diameter (for example, 25 mm) than the respiratory conduit received by the first retaining space 302 (for example, 22 mm). The narrower conduit is able to pass through each of the opening 306, second retaining space 304 and waist region 906 to be received by the first retaining space 302, while the relatively larger respiratory conduit is able to pass through the opening 306 into the second retaining space 304, but further movement of the relatively larger respiratory conduit to the first retaining space 302 is inhibited by the narrowed waist region 906.

The illustrated example of second retaining member 106 is designed to retain only one of the two different conduits at a time (since the conduit will extend into the waist region 906 and, possibly, the other of the retaining spaces). In some examples, however, the waist region 906 may be elongated to avoid interference between the conduits retained within the first retaining space 302 and the second retaining space 304, so that both conduits may optionally be retained by the second retaining member 106 simultaneously, if desired.

The second retaining member 106 includes a ledge 908 configured to support the one or more respiratory therapy components. The ledge 908 extends from the pair of retaining arms 904 towards the interior spaces 302, 304. The ledge 908 is configured to engage an outer surface of the inspiratory conduit 1008. As best shown in FIG. 10, the ledge 908 is configured to engage a corrugation 1010 of the inspiratory conduit 1008. The ledge 908 may alternatively engage an undulation in an outer surface of an alternative respiratory conduit, including a spirally-wound respiratory conduit such as an AirSpiral™ conduit available from Fisher & Paykel Healthcare, and/or may grip onto an alternative respiratory conduit having a generally smooth outer surface which is neither corrugated nor undulating. In the case of a respiratory conduit with an undulating outer surface, the minimum outer diameter may be marginally smaller than the maximum outer diameter, depending on the depth of the undulations. In the case of a respiratory conduit having a smooth outer surface, the minimum outer diameter may be the same as the maximum outer diameter. The ledge 908 can be dimensioned to generally correspond with an outer surface of an inspiratory conduit 1008. In other examples, the ledge 908 may not necessarily correspond with an outer surface of a respiratory conduit, yet may still be capable of holding the respiratory conduit via an interference fit and/or frictional engagement.

The ledge 908 of the example second retaining member 106 extends substantially perpendicularly from the pair of retaining arms 904, and is at least in part tapered towards the interior spaces 302, 304.

FIG. 10 shows a sectioned view of the second retaining member 106 taken at B-B of FIG. 9, and additionally illustrates, in part, an inspiratory conduit 1008. The ledge 908 is dimensioned to generally correspond with a corrugation 1010 of the inspiratory conduit 1008. More specifically, the ledge 908 is shaped and dimensioned to correspond with a trough of the corrugation 1010 of the inspiratory conduit 1008. The ledge 908 has a generally parabolic profile in cross-section and projects from the retaining arm 904 a distance about equal to a depth of the corrugations 1010 of inspiratory conduit 1008.

The ledge 908 within the first retaining space 302 defines a width or diameter of the first retaining space 302 which is less than a maximum outer diameter 1002 of a corresponding portion of the inspiratory conduit 1008, such that a corrugation 1010 rests atop the ledge 908, supporting the inspiratory conduit 1008 on the second retaining member 106. The ledge 908 may define a width or diameter of the first retaining space 302 which is equal to or marginally larger than the minimum outer diameter 1004 of the inspiratory conduit 1008, so that the conduit 1008 is not deformed when retained by the second retaining member 106. In some examples, however, the width or diameter defined by the ledge 908 may be smaller than the minimum outer diameter 1004.

The ledge 908 is similarly configured to engage a respiratory conduit, albeit one of larger minimum outer diameter 1004 and maximum outer diameter 1002, in the second retaining space 304 (not shown in FIG. 10).

FIG. 10 illustrates a corrugated respiratory conduit. The second retaining member 106 may alternatively engage and retain a respiratory conduit having an undulating outer surface, with less pronounced peaks and troughs, and/or a respiratory conduit having a smooth, non-undulating, outer surface. A respiratory conduit with undulations may be formed, for example, by spiral winding of a hollow elongate member and a bead in a double-helix pattern, for example. AirSpiral™ respiratory conduits are an example of such a respiratory conduit. The ledge 908 may be sized and shaped to engage the undulations similarly to the corrugations 1010. That is, the ledge 908 extends into the troughs so that a peak of the undulations rests upon an upper surface of the ledge 908. Alternatively, or additionally, the ledge 908 and retaining spaces 302, 304 may be sized and shaped to compress the undulating respiratory conduit. A smooth respiratory conduit may be similarly retained by compression. In some examples, any of the corrugated, undulating or smooth respiratory conduits may alternatively be retained by interference with a connector, similar to the how the non-sealing nasal cannula assembly 802 is retained by the first retaining member 104.

The inspiratory conduit 1008 hangs substantially perpendicular to the second retaining member 106, coaxial with the respective retaining space, in a substantially linear configuration. That is, the respiratory conduit 140 may be retained in a substantially vertical upright position, as shown in FIG. 10. This can facilitate drainage of any condensate accumulated within the inspiratory conduit 1008 during use in delivery of humidified breathing gases during respiratory therapy. A vertical or substantially vertical configuration of the inspiratory conduit 1008 may also be advantageous in minimizing the footprint occupied by the inspiratory conduit 1008, thereby reducing storage space requirements and/or the likelihood of creating an obstruction or being inadvertently knocked, compared to a draped or coiled respiratory conduit. In some examples the second retaining member 106 may be angled upwardly or downwardly, and at least a portion of the inspiratory conduit 1008 may not necessarily be held in a precisely vertical upright position. That is, the inspiratory conduit 1008 may curve at or near the point at which it is being held by the ledge 908. However, fluids may still be able to drain from the inspiratory conduit 1008. It will also be appreciated that an inspiratory conduit 1008 may tend not to hang linearly and/or vertically due to having been coiled for packaging and storage prior to use (e.g., having some “memory”), and may instead be curvilinear. Hanging the inspiratory conduit 1008 from at or near one end may assist in allowing the inspiratory conduit 1008 to relax and straighten somewhat, or at least not induce further curvature in the inspiratory conduit 1008.

The third retaining member 108 of the example respiratory therapy accessory 100 is configured to retain a patient interface assembly for use in delivering breathing gases to a patient for invasive ventilation, non-invasive ventilation (NIV), or nasal high flow (NHF) therapy. The third retaining member 108 may be longer than the first retaining member 104 and the second retaining member 106. That is, the third retaining member 108 may protrude further from the body 102 than either of the first and second retaining members 106, 108, as best shown in FIG. 3.

The third retaining member 108 of respiratory therapy accessory 100 includes a hook 122, as best shown in FIG. 1 and FIG. 4. The third retaining member 108 extends downwardly from the body 102, then curves upwardly into the hook 122 at a distal end. That is, the third retaining member 108 terminates in the hook 122 at a distal end of the third retaining member 108. In some examples, the third retaining member 108 may omit the hook 122. For example, the third retaining member 108 may be a straight or curved beam that extends from the body 102. This may be advantageous if the respiratory therapy accessory is intended to be attached to a support in either orientation, for example. In other examples, the third retaining member 108 may include a plurality of hooks 122.

A supporting member in the form of strut 126 extends from the body 102 to the third retaining member 108, joining at about a midpoint thereof, to support the third retaining member 108. In some examples, the first retaining member 104 and/or the second retaining member 106 may be similarly supported by a supporting member such as the strut 126.

The third retaining member 108 can retain the patient interface assembly (not shown) that is to be coupled to the inspiratory conduit 1008 retained by the second retaining member 106 during therapy. The patient interface assembly can be a total face mask (configured to seal around the patient's eyes, nose and mouth), a full-face mask (configured to seal around the patient's nose and mouth), an oro-nasal mask (configured to seal with each of the patient's nose/nares and mouth), a nasal mask (configured to seal around the patient's nose), a nasal pillows mask (configured to seal with the patient's nares), a nasal cannula assembly, an endotracheal tube, a tracheostomy tube, or any other patient interface assembly.

For example, a full-face mask may be hung from the hook 122 by positioning the headgear of the full-face mask over the hook, with the full-face mask suspended below the hook. Alternatively, the hook 122 may be positioned within a plenum chamber formed by a seal of the full-face mask, at the apex or nasal bridge region of the mask, for example. In some examples, the hook 122 can alternatively or additionally be configured to retain packaging containing a patient interface assembly, such as a packaging of an endotracheal tube configured for invasive ventilation therapy. The packaging may include a plastic bag or container (for example, clamshell packaging) with a keyhole aperture which is configured to be received by the hook 122, by way of non-limiting example.

The hook 122 may optionally be used to retain other respiratory therapy components. In one example, a wye-piece, to which an expiratory conduit is attached, may be hung from the hook 122 by positioning a corner of the wye-piece over the hook, with the expiratory conduit optionally suspended below the wye-piece.

The hook 122 includes an enlarged portion 124 at its distal end. As shown in FIG. 4, the enlarged portion 124 extends inwardly towards the body 102 relative to an adjacent portion of the hook 122. The enlarged portion 124 of the hook 122 may prevent or at least reduce the likelihood of the patient interface assembly or packaging from inadvertently coming off the hook 122.

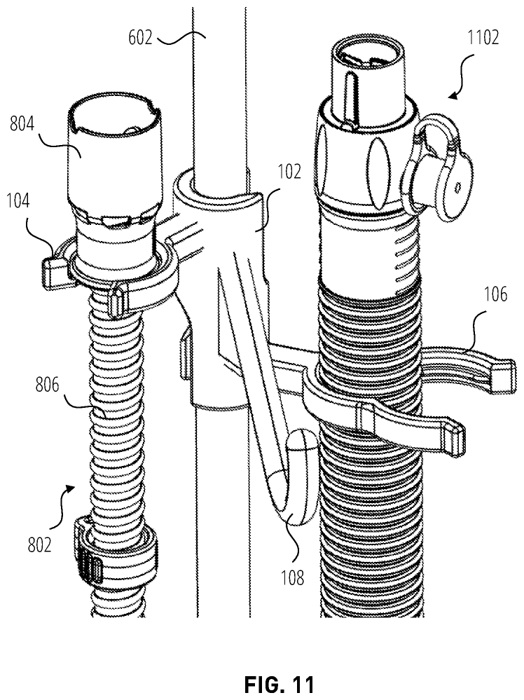

Referring to FIG. 11, respiratory therapy accessory 100 is shown in use, attached to a support 602 and retaining two respiratory therapy components.

The first retaining member 104 is shown retaining a respiratory therapy component in the form of a patient interface assembly configured for use for nasal high flow therapy, specifically the Optiflow™+nasal cannula assembly 802. The connector 804 and tubing 806 of nasal cannula assembly 802 are shown. A nasal cannula and attached headgear may hang from the opposing end of the tubing 806. A shoulder 808 of the connector is cradled by the first retaining member 104, as described above. The first retaining member 104 is also configured to alternatively retain at least an Optiflow™ 3S nasal cannula assembly, as described above. In other examples, the first retaining member 104 may be configured to retain any alternative patient interface assembly or respiratory therapy component.

The second retaining member 106 retains another respiratory therapy component in the form of a respiratory conduit configured to be coupled to a patient interface assembly suitable for non-invasive therapy. Specifically, the illustrated respiratory conduit is the corrugated inspiratory conduit 1102 which forms part of the RT232 Adult Optiflow™ Circuit Kit available from Fisher & Paykel Healthcare of Auckland, New Zealand. The inspiratory conduit 1102 is configured to be fluidly coupled with the connector 804 of the Optiflow™+nasal cannula assembly 802. The inspiratory conduit 1102 is retained by second retaining member 106 in the first retaining space 302 at least in part by ledge 908 engaging a corrugation of the inspiratory conduit 1102 as described above. However, the second retaining member 106 may be configured to receive and retain a range of 22 mm respiratory conduits, including the AirSpiral™ inspiratory conduit in the 850A61 F&P 850 Adult NIV and NHF Circuit Kit and the 950A61 F&P 950 Adult Bi-Level/CPAP Heated Circuit Kit, both also available from Fisher & Paykel Healthcare. These inspiratory conduits are not corrugated, but do have an undulating outer surface. Similarly, the second retaining space 304 (unoccupied in FIG. 11) may receive and retain a range of 25 mm respiratory conduits. An example is the inspiratory conduit of the RT319 F&P 850 Bi-level/CPAP Circuit Kit also available from Fisher & Paykel Healthcare.

The nasal cannula assembly 802 and the inspiratory conduit 1102 are each held by the respiratory therapy accessory 100 in a substantially linear and/or vertical configuration, which may provide one or more of the advantages of facilitating drainage of condensate within the tubing, relaxing the tubing which may have been coiled for packaging and storage, avoiding contact with contaminated or unhygienic surfaces, and minimizing a footprint of the respiratory therapy accessory 100 and retained respiratory therapy components.

The third retaining member 108 can retain a patient interface assembly (not shown) and/or packaging (not shown) containing a patient interface assembly, as described above.

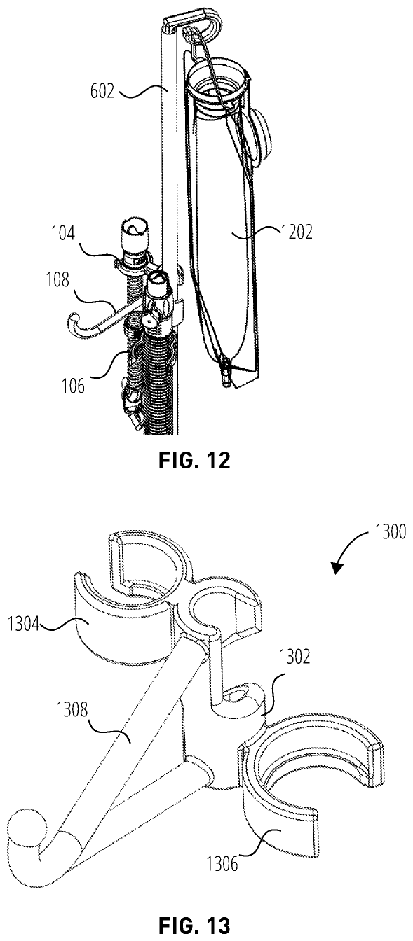

Referring to FIG. 12, in some examples the support 602 itself may also be configured to retain a respiratory therapy component, such as a sterile water bag 1202 for delivering humidification fluid to a humidifier, by way of non-limiting example.

The retaining members of respiratory therapy accessories according to the present disclosure may extend from the body in directions which span an angle of 180° or less, 140° or less, 120° or less, or 90° or less in some examples. By way of non-limiting example, the first retaining member 104 and second retaining member 106 of the respiratory therapy accessory 100 define an angle of about 120° when viewed along the longitudinal axis 202 as shown in FIG. 3. That is, the angle, for example of about 140°, is defined between a first axis that passes through the stem 706 of the first retaining member 104 and a second axis that passes through the stem 902 of the second retaining member 106. In this example, the first and second axes (as well as a third axis passing through the third retaining member 108) intersect at the longitudinal axis of the body 102, since the retaining members 104, 106, 108 extend radially from the body 102. Alternatively, in some examples where the respective stems of the first retaining member 104 and the second retaining member 106 are omitted, the angle may be defined between a first axis that bisects the first retaining member 104 and a second axis that bisects the second retaining member 106, when viewed along the longitudinal axis 202. Alternatively, in some examples where one stem is present, the angle may be defined between an axis that bisects one of the retaining members and an axis that passes through a stem of the other retaining member. For example, in the case where stem 706 is omitted, the angle may be defined between an axis that bisects the first retaining member 104 and an axis that passes through the stem 902 of the second retaining member 106 (which may also bisect the second retaining member 106).

This provides room for the sterile water bag 1202 or other accessory to hang from the support 602 adjacent the respiratory therapy accessory 100, ameliorating interference from the retaining members 104, 106, 108 or any respiratory therapy components retained by the respiratory therapy accessory, as shown in FIG. 12. For example, the respiratory therapy accessory 100 and/or sterile water bag 1202 may be arranged so that the water bag is provided to a side of the body 102 generally opposing the third retaining member 108 and spine 116 as shown in FIG. 12. Moreover, this provides enough room for the respiratory therapy components to hang from the retaining members 104, 106, 108 while ameliorating the risk that they may come into contact with each other. The respiratory therapy accessory in some examples may also be configured to selectively rotate or slide with respect to the support 602, and the disclosed angular arrangement of the retaining members 104, 106, 108 allows the respiratory therapy accessory 100 to be rotated to accommodate the sterile water bag 1202 or other accessory adjacent the respiratory therapy component, and/or slid along the support 602 past the sterile water bag or other accessory to ameliorate interference or otherwise adjust the height of the respiratory therapy accessory.

### Second Example

FIG. 13 shows a second example of a respiratory therapy accessory 1300. The respiratory therapy accessory 1300 is generally similar to the respiratory therapy accessory 100 of the first example. For brevity, some of the differences are described in detail below. The description of the respiratory therapy accessory 100 of the first example may otherwise apply equally to the equivalent integers of this second example.

Like respiratory therapy accessory 100, the respiratory therapy accessory 1300 includes a body 1302 configured to be clipped onto a support by translation and rotation. A first retaining member 1304, second retaining member 1306 and third retaining member 1308 extend radially from the body 1302.

The first retaining member 1304 and second retaining member 1306 in this second example extend from the body 102 in directly opposing directions, when viewed along the longitudinal axis. The first retaining member 1304, second retaining member 1306 and third retaining member 1308 therefore extend in directions spanning an angle of 180° with respect to the longitudinal axis, when viewed along the longitudinal axis. The first retaining member 1304 and second retaining member 1306 are spaced from each other in the direction of the longitudinal axis.

The first retaining member 1304 and second retaining member 1306 of respiratory therapy accessory 1300 omit the stems of the respiratory therapy accessory 100. That is, the part annular portions of first retaining member 1304 and second retaining member 1306 extend directly from the body 1302. The respiratory therapy accessory 1300, and any respiratory therapy components retained by the respiratory therapy accessory 1300, may therefore occupy a relatively smaller footprint when compared to respiratory therapy accessory 100. Torque created by the weight of respiratory therapy components acting upon the first retaining member 1304 and second retaining member 1306 may be reduced.

The first retaining member 1304 and second retaining member 1306 in this second example omit the outwardly diverging distal extensions of the first example.

The second retaining member 1306 of respiratory therapy accessory 1300 is configured to directly retain a single respiratory conduit, or a range of respiratory conduits of the same or similar maximum outer diameter and/or minimum outer diameter. However, the respiratory conduit may form part of a dual-limb circuit including an inspiratory conduit, expiratory conduit and a wye-piece fluidly coupling the inspiratory and expiratory conduits. The dual limb circuit may be retained by the respiratory therapy accessory 1300 by engaging one of the inspiratory conduit or expiratory conduit with the second retaining member 1306.

### Third Example

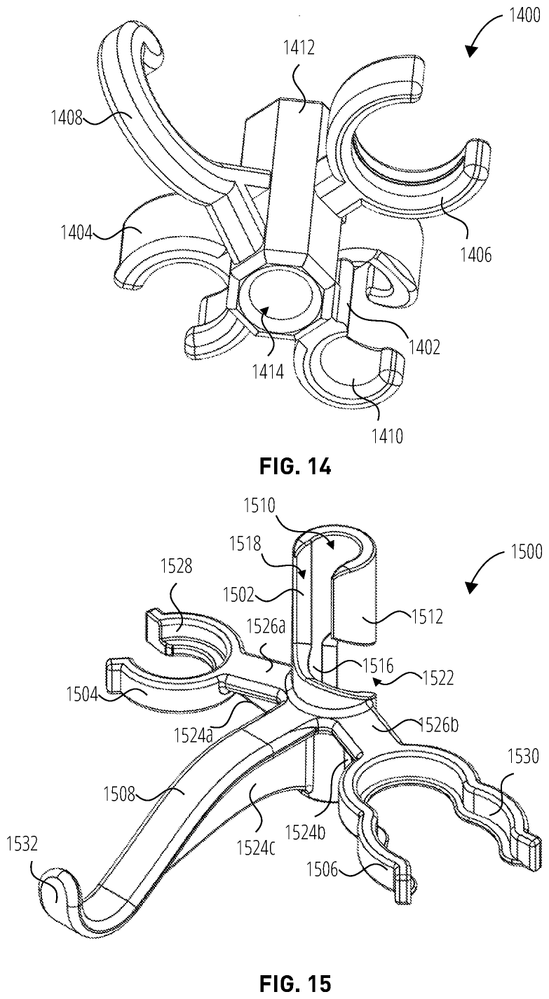

FIG. 14 shows a respiratory therapy accessory 1400 according to a third example. This third example is generally similar to the respiratory therapy accessory 100 and the respiratory therapy accessory 1300. For brevity, some of the differences are described in detail below. The description of the respiratory therapy accessory 100 of the first example and the respiratory therapy accessory 1300 of the second example may otherwise apply to the equivalent integers of this third example.

The respiratory therapy accessory 1400 includes a body 1402 configured to be attached to a support. A first retaining member 1404, second retaining member 1406 and third retaining member 1408 extend from the body 1402.

The body 1402 in this third example includes a first hollow structure 1410 and a second hollow structure 1412, attached or integrally formed side-by-side with parallel longitudinal axes.

As evident in FIG. 14, the first hollow structure 1410 of the body 1402 is similar to body 102 of the first example, and is similarly configured to clip onto a support by translation and rotation. Other attachment mechanisms may be used in alternative examples, however.

The second hollow structure 1412 of the body 1402 has a hexagonal outer cross-section, and a cylindrical interior cross-section. The hexagonal outer cross-section may be easier for a user to grip than a circular cross-section, and/or visually distinguishes the first hollow structure 1410 and the second hollow structure 1412. The second hollow structure has an aperture 1414 at a first (lower) end but may be closed at the opposing (upper) end (not shown). The aperture 1414 is configured to receive a support into the interior of the second hollow structure 1412. An entry to the aperture 1414 may be chamfered to guide a support into the second hollow structure 1412. In use, the respiratory therapy accessory 1400 may optionally be mounted to the top of a support, as an alternative to using the first hollow structure 1410. The respiratory therapy accessory 1400 may be slid down from the top of a support, until the support engages the closed upper end of the second hollow structure 1412.

The aperture 1414 and interior of the second hollow structure 1412 are suitable for engaging a cylindrical support, and may also be suitable for engaging certain other supports having non-circular cross-sections, such as a hexagonal cross-section. In other examples, however, the aperture 1414 and second hollow structure 1412 may be configured for attachment to other supports, including supports having rectangular, square, elliptical, or polygonal cross-sections, by having a corresponding internal shape.

In some examples, the first hollow structure 1410 and second hollow structure 1412 may have different internal diameters or cross-sections to allow use of the respiratory therapy accessory 1400 with different supports.

The first retaining member 1404, second retaining member 1406 and third retaining member 1408 of respiratory therapy accessory 1400 each extend from the second hollow structure 1412 of the body 1402. This advantageously spaces the retaining members and any retained respiratory therapy components further from the support and/or sterile water bag 1202 when the respiratory therapy accessory 1400 is attached to the support using the first hollow structure 1410. The third retaining member 1408 is diametrically opposed to the first hollow structure 1410. The first retaining member 1404 and the second retaining member 1406 are diametrically opposed to each other. The first retaining member 1404 and the second retaining member 1406 are both spaced an equal distance from one end of the body 1402.

### Fourth Example

FIG. 15 to FIG. 19 show a respiratory therapy accessory 1500 according to a fourth example. The respiratory therapy accessory 1500 is generally similar to the respiratory therapy accessory 100 of the first example. Like the first example, respiratory therapy accessory 1500 of this fourth example includes a body 1502 configured to be attached to a support. A first retaining member 1504, a second retaining member 1506 and a third retaining member 1508 extend from the body 1502. The body 1502 has a generally cylindrical hollow shape defining an interior space 1510, and comprises a first arm 1512 and a second arm 1514 extending arcuately from a spine 1516 to define a first opening 1518 and a second opening 1520 in the body 1502. An angled slot 1522 extends between, and connects, the first opening 1518 and the second opening 1520.

For brevity, some of the differences with respect to respiratory therapy accessory 100 of the first example are described in detail below. The description of the respiratory therapy accessory 100 may otherwise apply to the equivalent integers of this fourth example.

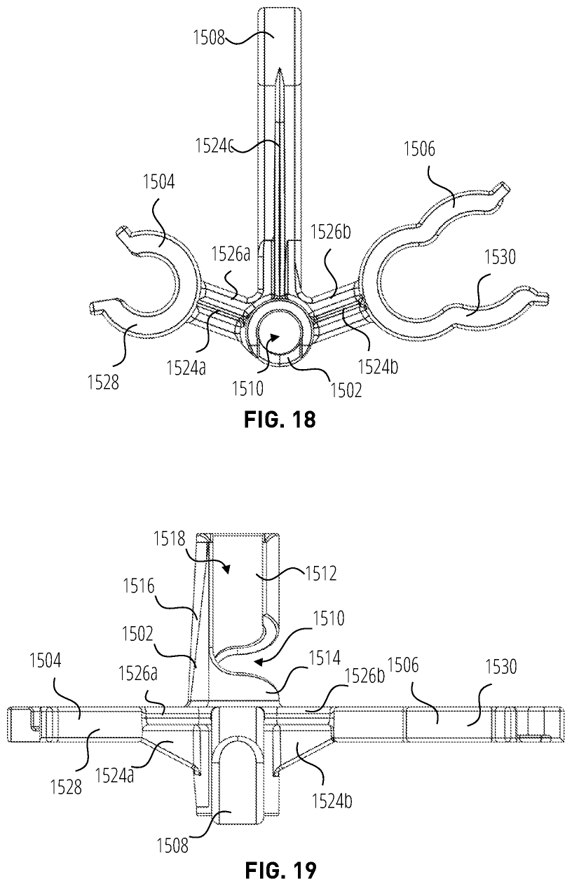

In this fourth example, the body 1502 has effectively been rotated 90° clockwise about the longitudinal axis of the body 1502 relative to the retaining members, when compared to the first example. That is, the first retaining member 1504 extends from the spine 1516 of the body 1502, on a side of the body generally opposing the slot 1522; the second retaining member 1506 extends from the second arm 1514, generally on the same side of the body 1502 as the slot 1522; and the third retaining member 1508 (being the longest of the retaining members) extends from the second arm 1514 of the body 1502, generally on the same side of the body 1502 as the first opening 1518 and generally opposing the second opening 1520. That is, the third retaining member 1508, the first opening 1518, the second opening 1520 and the longitudinal axis are aligned with each other when the respiratory therapy accessory 1500 is viewed from above or below (as shown in FIG. 18). In other words, the third retaining member 1308, the first opening 1518 and the second opening 1520 of the respiratory therapy accessory 1500 are each aligned on, or bisected by, a single plane.

It can be observed from FIG. 15 to FIG. 17 and FIG. 19 that the first retaining member 1504, the second retaining member 1506 and the third retaining member 1508 of this fourth example are each spaced the same distance from an upper end of the body 1502 in the direction of a longitudinal axis of the body 1502. That is, the retaining members 1504, 1506, 1508 are each positioned towards the lower end of the body 1502 and are not spaced apart in the axial direction as in the first example. The first arm 1512 at the upper end of the body 1502 is therefore free from any retaining members, and the first arm 1512 may therefore be less likely to inadvertently disengage from a support upon application of a force to any one of the retaining members, in use. However, in other examples, one or more of the retaining members may be spaced apart from another retaining member in the axial direction of the body 1502, and/or may extend from the first retaining member 1504 as shown in the first example, for instance.

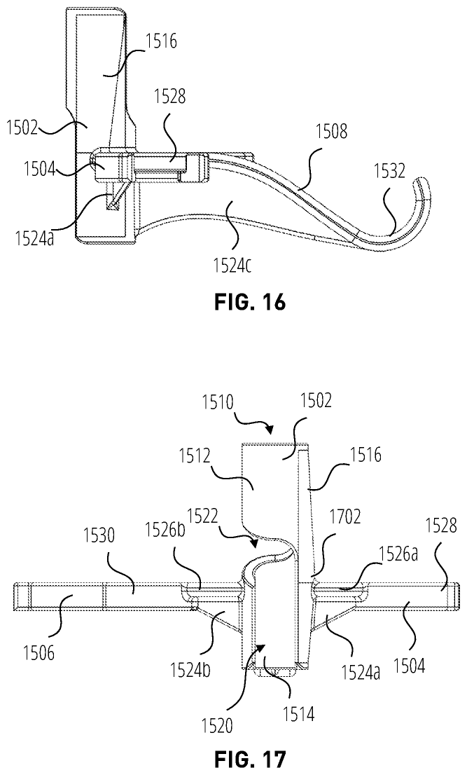

Each of the first retaining member 1504, the second retaining member 1506 and the third retaining member 1508 of this fourth example are supported by a supporting member in the form of respective gussets 1524a, 1524b, 1524c as shown in FIG. 15 to FIG. 19. Each of the gussets 1524a, 1524b, 1524c are substantially planar members arranged radially in respective vertical planes passing through the longitudinal axis of the body 1502. The gussets 1524a, 1524b, 1524c are each thinner than the respective stem 1526a, 1526b or third retaining member 1508, and thereby form a ‘T’-shaped vertical cross-section therewith. A height of each of the gussets 1524a, 1524b, 1524c (that is, the vertical distance between upper and lower edges of the respective gusset) tapers outwardly from the body 1502, as best shown in FIG. 16 and FIG. 17. At least a portion of the gusset 1524c may curve towards the underside of the third retaining member 1508.

Referring to FIG. 17, the gusset 1524a of the first retaining member 1504 extends between the part annular portion 1528, the underside of the first stem 1526a, and the body 1502. More specifically, the gusset 1524a of the first retaining member 1504 extends between a proximal end of the part annular portion 1528, a lower surface of the first stem 1526a and the body 1502 (e.g., the spine 1516) below the stem 1526a. The gusset 1524a has a substantially triangular shape.

Referring still to FIG. 17, the gusset 1524b of the second retaining member 1506 extends between a proximal end of the retaining arms 1530, a lower surface of the second stem 1526b and the body 1502 (e.g., the second arm 1514) below the stem 1526b. The gusset 1524b also has a substantially triangular shape, similar to the gusset 1524a.

Referring back to FIG. 16, the gusset 1524c of the third retaining member 1508 extends between a lower surface of the third retaining member 1508 and the body 1502 (e.g., the second arm 1514) below the third retaining member 1508. The gusset 1524c terminates at, or adjacent, the lowest point of the hook 1532 at the distal end of the third retaining member 1508. A lower edge of the gusset 1524c is contoured so that an overall height of the gusset 1524c tapers substantially continuously from the body 1502 towards the hook 1532. In other examples, it may be that portions of one or more of the gussets 1524a, 1524b, 1524c are tapered, or one or more of the gussets may not necessarily be tapered at all.

In some examples, one or more of the gussets 1524a, 1524b, 1524c may extend from an upper side of a stem or retaining member to the outer surface of the body 1502 immediately above the stem or retaining member. In other examples one or more of the gussets 1524a, 1524b, 1524c may be omitted. For example, either or both of the gussets 1524a, 1524b may be omitted in examples omitting either or both of the stems 1526a, 1526b.

The gussets described herein may reinforce the respective retaining members and reduce the likelihood of damage or breakage due to application of excessive force upon the retaining member, and/or allow for a reduction in the cross-section of the stems or the third retaining member. By way of example, the cross-sectional area of each of the stems 1526a, 1526b and third retaining member 1508 of this fourth example, measuring about 48 mm², are reduced in comparison with equivalent sections of the first example shown in FIG. 1, which may measure about 72 mm². The stems 1526a, 1526b and third retaining member 1508 also have a higher width-to-thickness ratio. For example, the third retaining member 1508 has a width-to-thickness ratio, in cross-section, of about 3:1, but in other examples may be between about 2:1 and 4:1. By way of comparison, the third retaining member 108 of the first example may have a width-to-thickness ratio of around 1.15:1. It is to be appreciated that the other examples described and illustrated herein may be modified to similarly include gussets.

Referring in particular to FIG. 15 and FIG. 16, the third retaining member 1508 (or at least an upper surface thereof) initially extends substantially perpendicularly from the body 1502, then curves downwardly before curving in an upward direction to terminate in a hook 1532 at a distal end of the third retaining member 1508. This arrangement may advantageously encourage a patient interface assembly or product packaging retainer by the third retaining member 1508 to settle at the low point of the third retaining member 1508, near the distal end, spacing the patient interface assembly or product packaging from the first retaining member 1504 and/or the second retaining member 1506. In some examples, the third retaining member 1508 may omit the hook 1532. In some examples, the third retaining member 1508 may not initially extend perpendicularly from the body 1502. For example, the third retaining member 1508 may extend downwardly directly from the body 1502, like the third retaining member 108 of the first example as shown in FIG. 1 and FIG. 4.

The third retaining member 1508 is shaped such that a width (in a horizontal direction, as illustrated) of the third retaining member 1508 is greater than a thickness (in a vertical direction, as illustrated) of the third retaining member 1508. That is, the third retaining member 1508 may have a generally horizontal flattened rectangular or elliptical cross-section. The third retaining member 1508, and in particular the hook 1532, may be configured to be received within a “euro slot” hang tab (or similar aperture) formed in product packaging. In particular, the third retaining member 1508 may have a horizontally-elongate cross-section with parallel upper and lower surfaces and rounded ends with a radius equal to half the thickness of the third retaining member 1308. The gusset 1524c may advantageously prevent or inhibit movement of the product packaging towards the proximal end of the third retaining member 1508 (e.g., beyond a mid-point of the third retaining member 1508) to maintain a spacing between the product packaging and the first retaining member 1504 and the second retaining member 1506. The hook 1532 may further be configured to retain other respiratory therapy components, such as a mask, as described above with respect to the first example, for instance.

Referring again to FIG. 17, the body 1502 of this fourth example comprises a thickened region 1702 which is thicker than adjacent portions of the body 1502 in the circumferential direction. The thickened region 1702 is located on the spine 1516, between the first arm 1512 and the second arm 1514. As illustrated, the thickened region 1702 of respiratory therapy accessory 1500 is located generally opposite the slot 1522. The thickened region 1702 in this fourth example varies in thickness in both the longitudinal and circumferential directions. That is, the thickness of the body 1502 in the thickened region 1702 is not uniform in either of the longitudinal or circumferential directions, but is thicker than adjacent regions of the body 1502 outside the thickened region 1702 in at least the circumferential direction. In the fourth example, the thickened region 1702 is thickest where the first retaining member 1504 extends from the body 1502. In the fourth example, the thickened region 1702 tapers continuously from this thickest region adjacent the first retaining member 1504 towards the opposing ends of the body 1502, but in other examples the thickened region may include one or more abrupt step-changes in thickness in the direction of the longitudinal axis. The thickened region 1702 extends substantially the full length of the body 1502 in the longitudinal direction, but extends partway around the circumference of the body 1502. However, in some examples the thickened region 1702 may extend partway along the full length of the body 1502 in the longitudinal direction. For example, the thickened region 1702 may extend about halfway from the first retaining member 1504 towards each end of the body 1502. The thickened region 1702 similarly tapers smoothly in the circumferential direction, but may alternatively include one or more abrupt changes in thickness.

The thickened region 1702 may strengthen the body 1502 to compensate for removal of material to create the slot 1522 and/or ameliorate the effects of material creep, ensuring that the body 1502 is sufficiently rigid to securely engage a support, in use.

The thickened region 1702 in this fourth example is defined by variations in the profile of the outer surface of the body 1502. That is, the inner surface of the body 1502 remains substantially cylindrical for stable engagement with a support 602, in use.

Referring to FIG. 18, the first retaining member 1504, second retaining member 1506 and third retaining member 1508 extend in directions spanning an angle of less than 180°, such as about 140°, with respect to the longitudinal axis, when viewed along the longitudinal axis. The third retaining member 1508 bisects the angle defined between the first retaining member 1504 and the second retaining member 1506. That is, the acute angle between the first retaining member 1504 and the third retaining member 1508 is equal to the acute angle between the second retaining member 1506 and the third retaining member 1508.

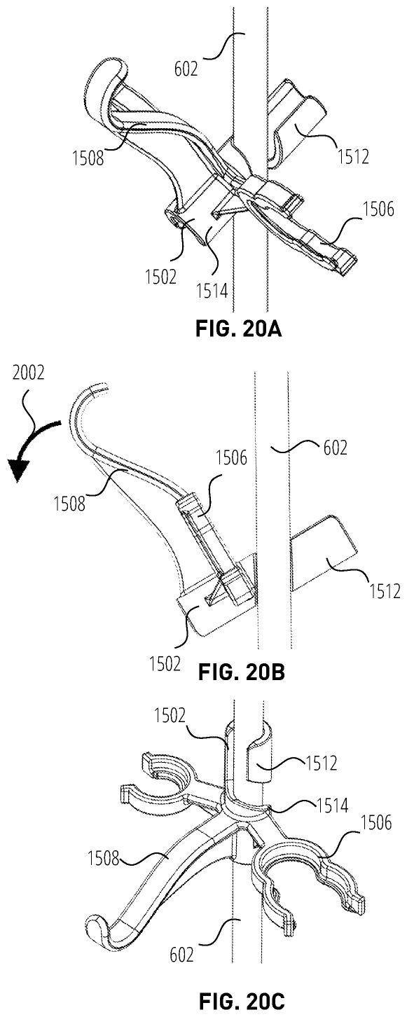

FIG. 20A to FIG. 20C illustrate the respiratory therapy accessory 1500 of the fourth example in various stages of engagement with a support 602, in use. Engagement of the respiratory therapy accessory 1500 is similar to engagement of the respiratory therapy accessory 100 as described above with respect to FIG. 6A to FIG. 6D. Briefly, however, the respiratory therapy accessory 1500 is firstly rotated to align the slot 1522 with the support 602 and translated so that the support 602 is received by the slot 1522 as shown in FIG. 20A, partially within the interior space 1510.

To engage the first arm 1512 and second arm 1514 with the support 602 to securely attach the body 1502 to the support 602, the body 1502 is rotated in the direction indicated by arrow 2002 in FIG. 20B. Rotation may be effected by pulling downwardly on the third retaining member 1508 in the direction of the arrow 2002. That is, the third retaining member 1508 effectively acts as a lever to cause rotation of the body 1502 which may provide a mechanical advantage during engagement of the first arm 1512 and second arm 1514 with the support 602. The respiratory therapy accessory 1500 rotates about a rotation axis which is substantially perpendicular to the third retaining member 1508 and/or the longitudinal axis of the body 1502. The rotation axis may pass through the slot 1522.

Rotation of the respiratory therapy accessory 1500 causes the first arm 1512 and the second arm 1514 to elastically deform about the circumference of the support 602, then recover to ‘snap’ the respiratory therapy accessory 1500 onto the support 602 and secure it in place with the support 602 further (e.g., fully) received within the interior space 1510 as shown in FIG. 20C.

To remove the respiratory therapy accessory 1500 from the support 602, the above steps may be reversed. That is, the first arm 1512 and the second arm 1514 are first disengaged from the support 602 by moving the third retaining member 1508 in an upwards direction (opposite to the direction of the arrow 2002 shown in FIG. 20B) to rotate the third retaining member 1308 about the axis of rotation, then translating the respiratory therapy accessory 1500 to the side so that the support 602 exits the interior space 1510 via the slot 1522.

### Fifth Example

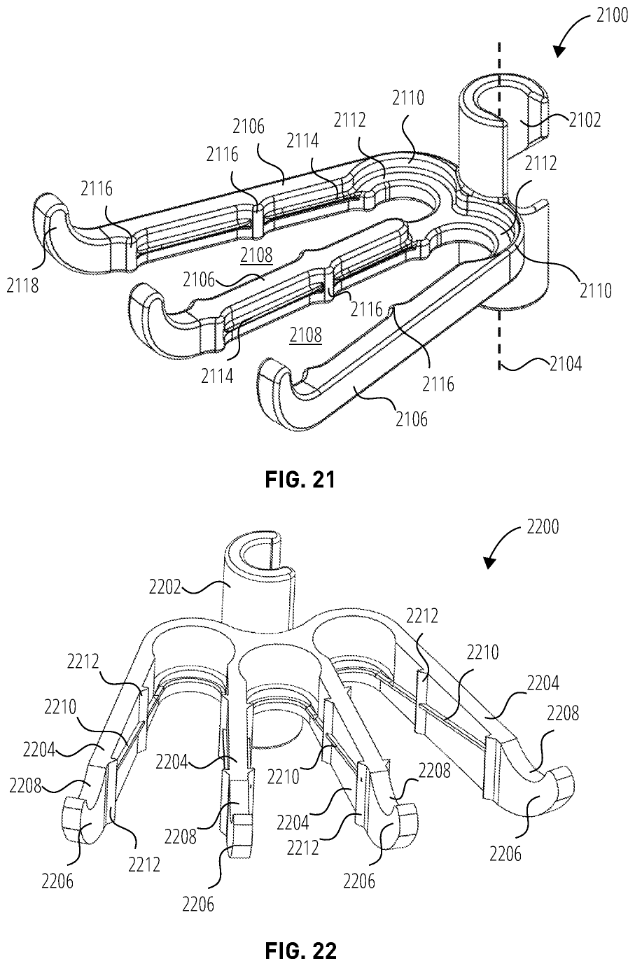

FIG. 21 shows a respiratory therapy accessory 2100 according to a fifth example. The respiratory therapy accessory 2100 includes a body 2102 configured to be coupled to a support 602. The body 2102 has a longitudinal axis 2104. The body 2102 is similar to body 102 as described above in relation to respiratory therapy accessory 100, and is similarly configured to clip onto a support by translation and rotation. Other attachment mechanisms may be used in alternative examples, however.

The respiratory therapy accessory 2100 includes a plurality of elongate arms 2106, in this particular example three elongate arms 2106, diverging away from the body 2102. In other examples, the respiratory therapy accessory 2100 may have fewer or more than three elongate arms 2106, for example, two arms, or four or more arms. In this fifth example, the elongate arms 2106 are, collectively, diametrically opposed to the slot of the body 2102.

The central elongate arm 2106 of respiratory therapy accessory 2100 extends substantially radially with respect to the longitudinal axis 2104. However, the two outer elongate arms 2106 extend from the body 2102 in a non-radial direction in this fifth example.

The angle of divergence between each of the two adjacent pairs of the three elongate arms 2106 in this fifth example are equal. That is, the central elongate arm 2106 bisects the two outer elongate arms 2106. In some examples, however, the angle between one pair of elongate arms 2106 may differ from an angle between at least one other pair of elongate arms 2106.

The plurality of elongate arms 2106 in this fifth example span an angle of less than 90°. That is, the outermost elongate arms 2106 of the plurality of elongate arms 2106 span an angle of less than 90°. In some examples, however, the plurality of elongate arms 2106 may span an angle of less than 120°, less than 150°, or equal to or less than 180°. That is, the outermost elongate arms of the plurality of elongate arms 2106 may span an angle equal to or less than 180°.

The angle spanned by the elongate arms 2106 is measured as described above with respect to the first example. Since the elongate arms 2106 of this fifth example may not all extend radially from the body 2102, however, it will be appreciated that the axes passing through the elongate arms do not intersect at the longitudinal axis of the body 2102. In some variants of this fifth example, it is also possible that the axes passing through the elongate arms 2106 may not all intersect at a common point. In such a case, the angle is to be measured between the axes of the two outermost elongate arms 2106.

The plurality of elongate arms 2106 in this fifth example extend perpendicularly from the body 2102 when viewed from the side. That is, the plurality of elongate arms 2106 extend from the body 2102 in a plane which is perpendicular to the longitudinal axis 2104 of the body 2102. If the longitudinal axis 2104 is vertical, the elongate arms 2106 will therefore extend substantially horizontally. In some examples, however, the plurality of elongate arms 2106 may extend from the longitudinal axis 204 of the body 2102 at an angle that is not perpendicular to the longitudinal axis 2104. That is, the elongate arms 2106 may be angled upwardly or downwardly, and/or may be curved.

An interior space 2108 is defined between each of the adjacent pairs of the three elongate arms 2106. A part annular portion 2110 is defined by proximal ends of an adjacent pair of elongate arms 2106. Each of the part annular portions 2110 defines a first, proximal, portion of the respective interior space 2108.

The part annular portion 2110 has a part annular ledge 2112. The part annular ledge 2112 can be configured to support a respiratory therapy component. The respiratory therapy component can be a patient interface assembly for delivering breathing gases to a patient. The patient interface assembly can be configured for use in nasal high flow therapy. The patient interface assembly may be a non-sealing nasal cannula assembly. The nasal cannula assembly may include a nasal cannula with prongs for insertion into the patient's nares, tubing fluidly coupled with the nasal cannula at one end to deliver a flow of breathing gases to a patient via the nasal cannula, and a connector attached to an opposing end of the tubing for coupling with a respiratory conduit configured to deliver the flow of breathing gases to the tubing from a respiratory humidifier, ventilator or the like. The humidifier can be integrated with a blower. The part annular ledge could alternatively, or additionally, be configured to support a respiratory conduit.

The illustrated part annular ledge 2112 of respiratory therapy accessory 2100 is configured to abut a shoulder of the connector of the respiratory conduit, similar to ledge 714 described above in relation to respiratory therapy accessory 100 with particular reference to FIG. 8. The part annular portions 2110 and part annular ledges 2112 are similarly configured to receive and retain the connector of a nasal cannula assembly. The nasal cannula assembly may be an Optiflow™+ or an Optiflow™ 3S, for example. In other examples, the part annular portions 2110 may each be configured to retain different respiratory therapy components. That is, the part annular portions 2110 may differ in size and/or shape.

The part annular ledge 2112 is spaced from a top surface of the part annular portion 2110. An arcuate wall 716 is defined between the part annular ledge 2112 and the top surface of the part annular portion 2110, similar to the arcuate wall 716 of the respiratory therapy accessory 100. The arcuate wall extends through an angle of greater than 180°. The arcuate wall is configured to partially surround, and reduce lateral movement of, the connector in use. The connector may be cradled by the combination of the part annular ledge 2112 and arcuate wall, similarly to what is shown in FIG. 8.

In some examples, the part annular ledge 2112 may be positioned halfway between the top surface of the part annular portion 2110 and a bottom surface of the part annular portion 2110. In some examples, the part annular ledge 2112 forms the bottom surface of the part annular portion 2110. In other examples, the part annular ledges 2112 may be omitted and the connector may be configured to abut a top surface of the part annular portion 2110.

The elongate arms 2106 of respiratory therapy accessory 2100 each include a linear ledge 2114 configured to support, at least in part, one or more respiratory therapy components, in particular one or more respiratory conduits. For example, the elongate arms 2106 may be configured to hold a first respiratory conduit having a first maximum outer diameter and/or minimum outer diameter, and a second respiratory conduit having a second maximum outer diameter and/or minimum outer diameter which is smaller than the corresponding first diameter of the first respiratory conduit. In another example, a first respiratory conduit may be suitable for use for adults or NIV therapy, and a second respiratory conduit of relatively smaller maximum outer diameter and/or minimum outer diameter 1004 may be suitable for use for infants and/or pediatric patients, or adult NHF therapy. The respiratory conduit can be part of a dual limb circuit including an inspiratory conduit, an expiratory conduit and a wye-piece.

The linear ledge 2114 extends from adjacent the part annular portion 2110 towards a distal end of the respective elongate arm 2106. In other examples the ledge need not necessarily be linear, but may be curvilinear for example.

The linear ledge 2114 can be configured to engage a corrugation or undulation of a respiratory conduit, or the linear ledge 2114 may be configured to grip a respiratory conduit having a relatively smooth outer surface. The linear ledge 2114 can be dimensioned to generally correspond with an outer surface of at least one respiratory conduit. That is, the linear ledge 2114 may have a generally parabolic cross-section and extend from the elongate arm 2106 a distance generally corresponding to a depth of the corrugations in the respiratory conduit, such as the trough of the corrugation 1010 of the inspiratory conduit 1008 in FIG. 10, for example.

The linear ledges 2114 of respiratory therapy accessory 2100 extend perpendicularly from the respective elongate arm 2106. In some examples, however, the linear ledge 2114 may extend from the respective elongate arm 2106 at an acute angle. In some examples, the linear ledge 2114 may taper in a direction that is towards the distal end of the respective elongate arm 2106.

Divergence of the elongate arms 2106 may advantageously enable the respiratory therapy accessory 2100 to receive and retain a wide variety of respiratory conduits of different diameters.

The plurality of elongate arms 2106 of respiratory therapy accessory 2100 may each further include a protrusion 2116 extending into the respective interior space 2108. The protrusions 2116 may be configured to assist in retaining a respiratory conduit within a defined region of interior space 2108. The protrusions 2116 can prevent or at least reduce the likelihood of the respiratory conduit inadvertently becoming disengaged from the linear ledges 2114 and falling to the ground. The protrusions 2116 may prevent or at least reduce the likelihood of two respiratory conduits retained by the respiratory therapy accessory 2100 coming into contact with each other.

The protrusion 2116 may intersect the linear ledge 2114. The protrusion 2116 in respiratory therapy accessory 2100 is perpendicular with the linear ledge 2114. That is, the protrusion 2116 intersects a linear ledge 2114 at an angle of 90°. In some examples, however, the protrusion 2116 may be spaced from the linear ledge 2114 so that they do not necessarily intersect.

In this fifth example, the protrusions 2116 of respiratory therapy accessory 2100 extend into the interior space 2108 further than the linear ledges 2114. The protrusions 2116 are arranged in opposing pairs, defining a waist region therebetween which may be narrower than a diameter of a selected respiratory conduit. The respiratory conduit, being resiliently deformable, deforms and elastically recovers as it is forced through a waist region or waist regions. A corrugation of the respiratory conduit may be engaged by an opposing pair of linear ledges 2114 to retain the respiratory conduit between the respective pair of elongate arms 2106.

A distal end of an elongate arm 2106 (in this example, each of the elongate arms 2106) includes a hook 2118. The hook 2118 is configured to retain a respiratory therapy component, such as a patient interface assembly. The hook 2118 can be configured to retain the respiratory therapy component similarly to the hook 122 described above in relation to the third retaining member 108 of respiratory therapy accessory 100, with particular reference to FIG. 1 and FIG. 4. The respiratory therapy component can be a patient interface assembly configured for use in invasive, NIV or NHF therapy. The patient interface assembly can be a total face mask, a full-face mask, an oro-nasal mask, a nasal mask, a nasal pillows mask, a nasal cannula assembly, an endotracheal tube, a tracheostomy tube or any other patient interface assembly suitable for respiratory therapy. In some examples, the hook 2118 may be configured to alternatively or additionally retain a product packaging containing a patient interface assembly. The packaging may have an aperture which receives the hook 2118. A distal end of the hook 2118 can optionally include an enlarged portion as described above in relation to respiratory therapy accessory 100. The hook 2118 and/or elongate arm 2106 can further include a recessed portion (not shown) to locate the patient interface assembly and impede inadvertent movement of the patient interface assembly along the elongate arm 2106 towards the body 2102 and/or the distal end of the hook 2118.

### Sixth Example

FIG. 22 shows a sixth example of a respiratory therapy accessory 2200. This sixth example is generally similar to the respiratory therapy accessory 2100 of the fifth example. For brevity, some of the differences are described in detail below. The description of the respiratory therapy accessory 2100 of the fifth example may otherwise apply equally to the equivalent integers of this sixth example.

The respiratory therapy accessory 2200 includes a body 2202 for attachment to a support. Extending from the body 2202 in this sixth example are four elongate arms 2204. The elongate arms 2204 span an angle of less than 90° from an imaginary intersection of lines passing through the two outermost of the elongate arms 2204 (when viewed from above or below, along the longitudinal axis of the body 2202).

The elongate arms 2204 of respiratory therapy accessory 2200 each include hooks 2206 which define a recess 2208. The recesses 2208 may inhibit a respiratory therapy component draped over the hook 2206 from inadvertently sliding off the hook towards the body 2202 and/or off the distal end of the hook.

The linear ledges 2210 extend along the plurality of elongate arms 2204 at an angle. That is, the linear ledges 2210 are angled upwardly from a proximal end of the elongate arms 2204 towards the distal ends. This may be advantageous in ameliorating or avoiding a respiratory conduit inadvertently moving in the distal direction and potentially becoming dislodged. The linear ledges 2210 in this sixth example also extend around the part annular portions defined between the proximal ends of the elongate arms 2204.

Each of the protrusions 2212 of respiratory therapy accessory 2200 are asymmetric, providing a relatively shallower angle for entry of a respiratory conduit between an adjacent pair of elongate arms 2204, in a direction towards the body 2202, and a relatively steeper angle for removal of the respiratory conduit in a direction towards the distal ends of the adjacent pair of elongate arms 2204. The asymmetric protrusions 2212 may advantageously provide a relatively lower resistance to insertion of a respiratory conduit compared to removal of the respiratory conduit.

It is to be understood that one or more features of any one of the above examples may be combined with one or more features of the other examples. By way of example, the third retaining member 1508 of the fourth example may be substituted for the third retaining member 108 of the first example, the third retaining member 1308 of the second example or the third retaining member 1408 of the third example; the second retaining member 106 of the first example may be substituted for the second retaining member 1306 of the second example, the second retaining member 1406 of the third example, or the second retaining member 1506 of the fourth example; and/or two or more of the elongate arms 2106 of the fifth example or elongate arms 2204 of the sixth example may be substituted for the third retaining member 108 of the first example, the third retaining member 1308 of the second example, the third retaining member 1408 of the third example, or the third retaining member 1508 of the fourth example.

### Changing Forms of Respiratory Therapy

It may be desirable to periodically or intermittently change a patient between NIV and NHF therapies. For example, it has been found that intermittent use of NHF therapy can be beneficial for the recovery of patients receiving NIV therapy. Alternatively, or additionally, patient interface assemblies for NHF therapy are generally less intrusive than NIV interface assemblies, allowing the patient to eat and/or communicate more freely with medical professionals and visitors.

Use of the disclosed respiratory therapy accessories can advantageously reduce confusion or complication during the period in which the patient is changed or alternated between different forms of respiratory therapy. It can also advantageously avoid or ameliorate one or more of inconvenience in finding somewhere suitable to place various respiratory therapy components, the risk of respiratory therapy components falling onto the floor, and/or potential contamination. Use of the disclosed respiratory therapy accessories will be described in further detail below with reference to non-limiting example processes and the respiratory therapy accessory 100 of the first example.

In one example process, a patient can be changed or alternated between NIV therapy and NHF therapy. A gases source, such as a ventilator, may be used to deliver gases to the patient during NIV therapy. The same gases source may also have an NHF mode configured to provide NHF therapy.

The patient may already be on NIV therapy, wearing a first patient interface assembly configured for use for NIV therapy, and is to be transitioned to NHF therapy. The first patient interface assembly can be a total face mask (configured to seal around the patient's eyes, nose and mouth), a full-face mask (configured to seal around the patient's nose and mouth), an oro-nasal mask (sealing with or around each of the patient's nose/nares and mouth individually), a nasal mask (configured to seal around the patient's nose), a nasal pillows mask (configured to seal with the patient's nares), or any other patient interface assembly suitable for use in NIV therapy. The first patient interface assembly will generally be coupled with a single limb circuit (with an exhalation port in line with the first patient interface assembly, if the first patient interface assembly is not vented), but in some cases may be coupled with a dual limb circuit. Prior to transitioning to NHF therapy, the respiratory therapy accessory 100 is attached to a support. The first retaining member 104 of the respiratory therapy accessory 100, may hold a second patient interface assembly configured for use for NHF therapy. The second patient interface assembly can be a non-sealing nasal cannula assembly, such as the Optiflow™+nasal cannula assembly 802 or the Optiflow™3S nasal cannula assembly, for example. Alternatively, the third retaining member 108 of the respiratory therapy accessory 100 can hold a packaging containing the second patient interface assembly. In some cases, the second patient interface assembly is not necessarily held by the first retaining member 104, but instead may be placed near the patient, such as beside the patient's face or on the patient's bed.

To transition to NHF therapy, the first patient interface assembly can be removed from the patient's face and can be hung from the respiratory therapy accessory 100 for storage, such as from the hook 122 of the third retaining member 108. The second patient interface assembly can be removed from the first retaining member 104 and placed on the patient's face.

The second patient interface assembly will in most cases be used with a single limb circuit. The inspiratory conduit can be removed from the first patient interface assembly and, if necessary, temporarily held by the second retaining member 106 before it is attached to the second patient interface assembly (via an adapter, if necessary). In other cases, a different inspiratory conduit and/or a different gases source, may be used.

In some cases, the NIV circuit may be a dual limb circuit. The inspiratory conduit can be removed from the expiratory conduit and wye-piece and connected to the second patient interface assembly. The expiratory conduit and wye-piece may be hung from the second retaining member 106 of the respiratory therapy accessory 100.

In some cases, the NIV circuit and NHF circuit may use different inspiratory conduits. For example, the NIV circuit may be a dual limb circuit and the NHF circuit may be a single limb circuit, having different inspiratory conduits. In such cases, the NIV circuit and the NHF circuit would typically use different gases sources.

In some cases, both the NIV and NHF therapy may use dual limb circuits. The second patient interface assembly may be configured to be coupled, directly or indirectly, to the same dual limb arrangement as the first patient interface assembly, and therefore the same gases source. In such cases, the gases source may block an expiratory port to which the expiratory conduit couples, thus preventing gases returning to the gases source or bypassing the patient through the wye-piece.

The gases source, configured for use for NIV therapy, can be switched off or placed on stand-by after the first patient interface assembly is removed from the patient. The gases source, configured for use for NHF therapy, can be turned on prior to or after placing the second patient interface assembly over the patient's face. As noted above, the gases source can be configured for use for both NIV therapy and NHF therapy. In such examples, the gases source can be changed from a mode suitable for NIV therapy to a mode suitable for NHF therapy. In other cases, different gases sources may be used for the NIV and NHF therapies.

In other cases, the patient can already be on NHF therapy, wearing the second patient interface assembly, and is to be transitioned to NIV therapy. Prior to transitioning to NIV therapy, the third retaining member 108 can already be holding the first patient interface assembly or packaging containing the first patient interface assembly suitable for NIV therapy.

To transition to NIV therapy, the second patient interface assembly can be removed from the patient's face and hung on the first retaining member 104. The first patient interface assembly can then be placed over the patient's face. In some examples, a respiratory conduit, configured to deliver gases to the patient via the first patient interface assembly, can be coupled to the first patient interface assembly prior to or after placing the first patient interface assembly over the patient's face. In other examples, the first patient interface assembly can be coupled to a dual limb circuit. The dual limb circuit may be configured to be coupled to both the first and second patient interface assemblies. The gases source, configured for use for NHF therapy, can be switched off after removing the second patient interface assembly from the patient's face. The gases source, configured for use for NIV therapy, can be turned on prior to or after placing the first patient interface assembly over the patient's face. As noted above, in some examples, the gases source can be configured for use for both NIV therapy and NHF therapy. In such examples, the gases source can be changed from a mode suitable for NHF therapy to a mode suitable for NIV therapy.

In another example process, a patient can be changed or alternated between invasive ventilation therapy and NHF therapy. The patient can already be on invasive ventilation therapy, and is to be transitioned to NHF therapy. The patient can be fitted with a third patient interface assembly, such as an endotracheal tube by intubation, or a tracheostomy tube. The third patient interface assembly can be coupled to a dual limb circuit. Prior to transitioning to NHF therapy, the first retaining member 104 can hold the second patient interface assembly. Alternatively, the third retaining member 108 can hold a packaging containing a second patient interface assembly, such as the RT481 Post-Extubation Kit available from Fisher & Paykel Healthcare of Auckland, New Zealand. The second retaining member 106 can hold an inspiratory conduit configured to be coupled with the second patient interface assembly. For example, the second retaining member 106 may include the ledge 908 described above, which engages with a corrugation or undulation of the inspiratory conduit.

To transition from invasive ventilation therapy to NHF therapy, the patient can be extubated. That is, the third patient interface assembly is removed from the patient and can be disposed of. The second patient interface assembly can then be placed on the patient's face. The same inspiratory conduit of the dual limb circuit can be used during both invasive ventilation therapy and NHF therapy. The second patient interface assembly will usually be coupled with a single limb circuit. The inspiratory conduit may therefore be removed from the dual limb circuit and fluidly coupled with the second patient interface assembly, and the expiratory conduit and wye-piece placed on the respiratory therapy accessory 100. The third retaining member 108 can hold the wye-piece. Additionally and/or alternatively, the second retaining member 106 can hold the expiratory conduit. In some cases, however, the second patient interface assembly may be coupled to the same dual limb circuit that the third patient interface assembly couples with, in which case the third patient interface assembly can be replaced with the second patient interface assembly. The second patient interface assembly may alternatively be coupled to a second gases source via a different inspiratory conduit (via an adapter, if necessary). The second patient interface assembly may alternatively be coupled to the same gases source via a different inspiratory conduit. The different inspiratory conduit may be held ready on the respiratory therapy accessory 100.

From the foregoing, it will be appreciated that the disclosed respiratory therapy accessories may advantageously provide for convenient storage of various patient interfaces and/or respiratory conduits used in non-invasive therapy, nasal high flow therapy, and/or invasive ventilation therapy. The disclosed respiratory therapy accessories may facilitate transition between two different forms of respiratory therapy.

Respiratory therapy systems according to the disclosure may include a respiratory therapy accessory as described herein, and one or more of the support, gases source, humidifier, integrated gases source and humidifier, inspiratory conduit, expiratory conduit, and wye-piece.

### LISTING OF DRAWING ELEMENTS

100 respiratory therapy accessory
102 body
104 first retaining member
106 second retaining member
108 third retaining member
110 interior space
112 first arm
114 second arm
116 spine
118 first opening
120 opposing edges
122 hook
124 enlarged portion
126 strut
202 longitudinal axis
204 second opening
206 slot
208 rotation axis
210 rotation direction
302 first retaining space
304 second retaining space
306 opening
602 support
604 translation direction
606 rotation direction
702 part annular portion
704 interior space
706 stem
708 connector portion
710 tubing portion
712 opening
714 ledge
716 arcuate wall
802 nasal cannula assembly
804 connector
806 tubing
808 shoulder
902 stem
904 retaining arm
906 waist region
908 ledge
1002 maximum outer diameter
1004 minimum outer diameter
1006 heating element
1008 inspiratory conduit
1010 corrugation
1102 inspiratory conduit
1202 sterile water bag
1300 respiratory therapy accessory
1302 body
1304 first retaining member
1306 second retaining member
1308 third retaining member
1400 respiratory therapy accessory
1402 body
1404 first retaining member
1406 second retaining member
1408 third retaining member
1410 first hollow structure
1412 second hollow structure
1414 aperture
1500 respiratory therapy accessory
1502 body
1504 first retaining member
1506 second retaining member
1508 third retaining member
1510 interior space
1512 first arm
1514 second arm
1516 spine
1518 first opening
1520 second opening
1522 slot
1524a gusset
1524b gusset
1524c gusset
1526a stem
1526b stem
1528 part annular portion
1530 retaining arms
1532 hook
1702 thickened region
2002 rotation direction
2100 respiratory therapy accessory
2102 body
2104 longitudinal axis
2106 elongate arm
2108 interior space
2110 part annular portion
2112 part annular ledge
2114 linear ledge
2116 protrusion
2118 hook
2200 respiratory therapy accessory
2202 body
2204 elongate arm
2206 hook
2208 recess
2210 linear ledge
2212 protrusion

1-131. (canceled)

132. A respiratory therapy accessory comprising:
a body configured to be coupled to a support, the body comprising a first arm and a second arm;
a slot disposed between the first arm and the second arm, the slot configured to receive the support; and
a retaining member extending from the body and configured to retain a respiratory therapy component in use;
wherein the first arm and the second arm are configured to engage the support when the body is rotated about a rotation axis in a first direction; and
wherein the retaining member extends from the body in a direction which produces a torque on the body in the first direction.

133. (canceled)

134. The respiratory therapy accessory of claim 132, wherein the first arm and the second arm are part annular.

135-137. (canceled)

138. The respiratory therapy accessory of claim 132, wherein the retaining member comprises a third retaining member and the respiratory therapy accessory comprises a first retaining member and a second retaining member, wherein the first retaining member and the second retaining member extend from the body at an acute angle to the third retaining member, when viewed from above.

139-219. (canceled)

220. The respiratory therapy accessory of claim 138, wherein the first retaining member extends from the body on a side of the body opposing the slot.

221. The respiratory therapy accessory of claim 138, wherein the second retaining member extends from the second arm on a same side of the body as the slot.

222. The respiratory therapy accessory of claim 138, wherein the third retaining member is a longest one of the first retaining member, the second retaining member, and the third retaining member and extends from the second arm on a same side of the body as a first opening in the body and opposing a second opening in the body.

223. The respiratory therapy accessory of claim 138, wherein the first retaining member, the second retaining member, and the third retaining member are each positioned towards a lower end of the body.

224. The respiratory therapy accessory of claim 138, the body comprising a thickened region which is thicker than adjacent portions of the body in a circumferential direction, wherein the thickened region is thickest where the first retaining member extends from the body.

225. The respiratory therapy accessory of claim 224, wherein the thickened region tapers continuously from a thickest region adjacent the first retaining member towards opposing ends of the body.

226. The respiratory therapy accessory of claim 138, wherein each of the first retaining member, the second retaining member, and the third retaining member have a different shape whereby they are structurally dissimilar to each other.

227. The respiratory therapy accessory of claim 132, the body comprising a spine, wherein the first arm extends in a circumferential direction from one side of the spine, and the second arm extends in an opposing circumferential direction from an opposing side of the spine.

228. The respiratory therapy accessory of claim 227, wherein:
distal ends of the first arm and the second arm and opposing portions of the spine include substantially planar opposing edges at a respective first opening in the body and second opening in the body; and
the opposing edges are configured to determine, in part, a torque required to engage the first arm and the second arm with the support such that the support is received within an interior space of the body.

229. The respiratory therapy accessory of claim 132, wherein the first arm defines a first opening to an interior space of the body and the second arm defines a second opening to the interior space, wherein the retaining member, the first opening, and the second opening are aligned on a plane.

230. The respiratory therapy accessory of claim 132, comprising a support member supporting the retaining member, wherein the support member comprises a strut or a gusset.

231. The respiratory therapy accessory of claim 132, wherein the rotation axis is perpendicular to the retaining member.

232. The respiratory therapy accessory of claim 132, wherein the body is configured to couple with the support so that it may selectively slide along and rotate about the support.

233. A respiratory therapy system, comprising:
the respiratory therapy accessory of claim 132; and
the support.

234. The respiratory therapy system of claim 233, wherein the support forms at least part of any one or more of an intravenous bag support, a water bag pole, a roll stand, a ventilator trolley, or a bed frame.

235. A respiratory therapy accessory comprising:
a body configured to be coupled to a support, the body comprising a first arm and a second arm;
a slot disposed between the first arm and the second arm, the slot configured to receive the support;
a retaining member extending from the body and configured to retain a respiratory therapy component in use;
wherein the first arm and the second arm are configured to engage the support when the body is rotated about a rotation axis in a first direction;
wherein the retaining member extends from the body in a direction which produces a torque on the body in the first direction;
wherein the rotation axis is perpendicular to the retaining member;
wherein the body comprises a spine, wherein the first arm extends in a circumferential direction from one side of the spine, and the second arm extends in an opposing circumferential direction from an opposing side of the spine; and
wherein the first arm defines a first opening to an interior space of the body and the second arm defines a second opening to the interior space.

236. A respiratory therapy accessory comprising:
a body configured to be coupled to a support, the body comprising a first arm and a second arm;
a slot disposed between the first arm and the second arm, the slot configured to receive the support;
a first retaining member;
a second retaining member; and
a third retaining member extending from the body and configured to retain a respiratory therapy component in use;
wherein the first arm and the second arm are configured to engage the support when the body is rotated about a rotation axis in a first direction;
wherein the third retaining member extends from the body in a direction which produces a torque on the body in the first direction;
wherein the first retaining member and the second retaining member extend from the body at an acute angle to the third retaining member, when viewed from above; and
wherein each of the first retaining member, the second retaining member, and the third retaining member have a different shape whereby they are structurally dissimilar to each other.
